(19) United States
(12) Patent Application Publication
(10) Pub. No.: US 2024/0033375 A1
(43) Pub. Date: Feb. 1, 2024

(54) COMPOSITIONS USEFUL FOR TREATING SPINAL AND BULBAR MUSCULAR ATROPHY (SBMA)

(71) Applicant: The Trustees of the University of Pennsylvania, Philadelphia, PA (US)

(72) Inventors: Eileen Workman, Philadelphia, PA (US); Christian Hinderer, Baltimore, MD (US)

(21) Appl. No.: 18/485,352

(22) Filed: Oct. 12, 2023

Related U.S. Application Data
(63) Continuation-in-part of application No. PCT/US22/24415, filed on Apr. 12, 2022.
(60) Provisional application No. 63/381,938, filed on Nov. 2, 2022.
(60) Provisional application No. 63/415,610, filed on Oct. 12, 2022.
(60) Provisional application No. 63/293,505, filed on Dec. 23, 2021.
(60) Provisional application No. 63/187,883, filed on May 12, 2021.
(60) Provisional application No. 63/173,885, filed on Apr. 12, 2021.

(51) Int. Cl. A61K 48/00; A61P 25/00

(52) U.S. Cl. CPC A61K 48/0033; A61K 48/0075; A61P 25/00

(57) ABSTRACT

Compositions useful for treatment of Spinal and Bulbar Muscular Atrophy (SBMA) comprising administration of a recombinant adeno-associated virus (rAAV) vector having an AAV capsid and a vector genome comprising a sequence encoding at least one hairpin forming miRNA that comprises a targeting sequence which binds a target site on the mRNA of human androgen receptor, wherein the miRNA inhibits expression of human androgen receptor, is provided. Also provided are compositions containing a rAAV vector and methods of treating SBMA in patient comprising administration of a rAAV vector.

### CROSS REFERENCE TO RELATED APPLICATIONS

This application is a continuation-in-part of International Patent Application No. PCT/US22/24415, filed Apr. 12, 2022, which claims the priority of U.S. Provisional Application Nos. 63/293,505, filed Dec. 23, 2021, 63/187,883, filed May 12, 2021, and 63/173,885, filed Apr. 12, 2021. This application also claims priority of U.S. Provisional Application Nos. 63/381,938, filed Nov. 2, 2022, and 63/415,610, filed Oct. 12, 2022.

### REFERENCE TO AN ELECTRONIC SEQUENCE LISTING

The contents of the electronic sequence listing (23-10249.US Seq-Listing.xml; Size: 77,824 bytes; and Date of Creation: Oct. 11, 2023) is herein incorporated by reference in its entirety.

### BACKGROUND OF THE INVENTION

Spinal and Bulbar Muscular Atrophy (referred to herein as SBMA or Kennedy's Disease) is an X-linked, slowly progressive motor neuron disease caused by a polyglutamine (CAG) expansion tract within exon 1 of the androgen receptor (AR). The expansion results in the nuclear aggregation of the AR protein causing motor neuron degeneration almost exclusively in males due to androgen-mediated activation of toxicity. To date, no effective treatment has been approved for SBMA. Since knockdown of the androgen receptor in neurons is not known to result in adverse effects, lowering AR levels in SBMA is an attractive strategy for treatment of the disease.

Adeno-associated virus (AAV), a member of the Parvovirus family, is a small non-enveloped, icosahedral virus with single-stranded linear DNA (ssDNA) genomes of about 4.7 kilobases (kb) long. The wild-type genome comprises inverted terminal repeats (ITRs) at both ends of the DNA strand, and two open reading frames (ORFs): rep and cap. Rep is composed of four overlapping genes encoding rep proteins required for the AAV life cycle, and cap contains overlapping nucleotide sequences of capsid proteins: VP1, VP2 and VP3, which self-assemble to form a capsid of an icosahedral symmetry.

AAV is assigned to the genus, Dependovirus, because the virus was discovered as a contaminant in purified adenovirus stocks. AAV's life cycle includes a latent phase at which AAV genomes, after infection, are site specifically integrated into host chromosomes and an infectious phase in which, following either adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer.

What is desirable are therapeutics for treatment of SBMA.

### SUMMARY OF THE INVENTION

A therapeutic, recombinant (r), replication-defective, adeno-associated virus (AAV) is provided which is useful for treating and/or reducing the symptoms associated with SBMA in human patients in need thereof. The rAAV is desirably replication-defective and carries a vector genome expressing a miRNA targeting the androgen receptor to motor neurons.

In one aspect, an adeno-associated virus (AAV) is provided. The AAV includes an AAVhu68 capsid having packaged therein a vector genome. The vector genome includes an expression cassette comprising a nucleic acid sequence encoding at least one hairpin forming miRNA that comprises a targeting sequence that binds a miRNA target site on the mRNA of human androgen receptor. The miRNA coding sequence is operably linked to regulatory sequences which direct expression of the nucleic acid sequence in a subject. The miRNA inhibits expression of human androgen receptor. The vector genome comprises an AAV 5′ ITR; a CMV enhancer; a CBA promoter; a chimeric intron; SEQ ID NO: 4 or a sequence having up to 10 substitutions; a woodchuck post-regulatory element (WPRE); a poly A; and an AAV 3′ ITR.

In certain embodiments, the vector genome comprises one or more of a) the AAV 5′ ITR of SEQ ID NO: 34; b) the CMV enhancer of SEQ ID NO: 29; c) the CBA promoter of SEQ ID NO: 30; d) the chimeric intron of SEQ ID NO: 31; e) SEQ ID NO: 4; f) the woodchuck post-regulatory element (WPRE) of SEQ ID NO: 32; g) the RBG poly A of SEQ ID NO: 33; or h) the AAV 3′ ITR of SEQ ID NO: 35.

In one embodiment, the vector genome comprises the expression cassette of SEQ ID NO: 26, or a sequence sharing at least 80% identity therewith. In certain embodiments, the vector genome comprises SEQ ID NO: 28, or a sequence sharing at least 80% identity therewith.

In another aspect, a pharmaceutical composition is provided, comprising an AAV as described herein, and a pharmaceutically acceptable aqueous suspending liquid, excipient, and/or diluent.

In another aspect, a method for treating a subject having Spinal and Bulbar Muscular Atrophy (SBMA) is provided. The method includes delivering an effective amount of a pharmaceutical composition or AAV as described herein to a subject in need thereof.

In another aspect, an expression cassette comprising a nucleic acid sequence encoding at least one hairpin forming miRNA that comprises a targeting sequence that binds a miRNA target site on the mRNA of human androgen receptor is provided. The miRNA coding sequence is operably linked to regulatory sequences which direct expression of the nucleic acid sequence in a subject. The miRNA inhibits expression of human androgen receptor. The vector genome comprises an AAV 5′ ITR; a CMV enhancer; a CBA promoter; a chimeric intron; SEQ ID NO: 4 or a sequence having up to 10 substitutions; a woodchuck post-regulatory element (WPRE); a poly A; and an AAV 3′ ITR.

In certain embodiments, the expression cassette comprises one or more of a) the CMV enhancer of SEQ ID NO: 29; b) the CBA promoter of SEQ ID NO: 30; c) the chimeric intron of SEQ ID NO: 31; d) SEQ ID NO: 4; e) the woodchuck post-regulatory element (WPRE) of SEQ ID NO: 32; or f) the RBG poly A of SEQ ID NO: 33.

These and other aspects of the invention are apparent from the following detailed description of the invention.

### BRIEF DESCRIPTION OF THE DRAWINGS

FIG. 1A-FIG. 1D are 4 graphs showing that SBMA onset correlates with the number of CAG repeats.

FIG. 2A-FIG. 2C are 3 graphs showing that SBMA disease rate of progression is similar for all patients having <47 CAG or >47 CAG repeats. The graphs show the fraction of patients exhibiting each symptom vs age. The groups are divided into in patients with <47 repeats or >47 repeats.

FIG. 3A-FIG. 3D are 4 graphs showing that SBMA disease rate of progression is similar for all patients having <47 CAG or >47 CAG repeats. Time from first symptom (weakness) to need for handrail to ascend stairs, use of cane, wheelchair dependence, and death is highly reproducible between patients. Patients with >47 CAG repeats have earlier onset but identical progression.

FIGS. 4A-4B show the screening results of AR-targeting miRNAs in HEK293 cells. HEK293 cells were transfected with in vitro Block-iT plasmids. The Block-IT plasmids contain a CMV promoter, emGFP, cloning site for miRNA and TK polyA. miRNAs were designed using Block-iT online software. FIG. 4A shows the mRNA levels of the androgen receptor after knockdown of several individual miRNAs. FIG. 4B shows the protein levels of the androgen receptor after knockdown of several individual miRNAs. Both mRNA and protein data highlight miR 3610 an effective miRNA to knockdown the androgen receptor in vitro.

FIGS. 5A-5C show evaluation of administration in mice. In FIG. 5A neonatal mice were either injected with PBS or miR NeuN at 1e11 GC via ICV. Brains were harvested at day 14 and processed for Western blot analysis with NeuN antibody. β-actin was used as a loading control. FIG. 5B shows the quantification of protein as percentage of NeuN in each group. In FIG. 5C adult mice were injected with PBS, AAV.CB7.miR.NeuN or AAV.CB57.GFP at 3e11 GC via IV. Brains were harvested at day 14 and processed for Western blot analysis with NeuN antibody. The scatterplot graph shows the quantification of protein as percentage of NeuN in each group.

FIGS. 6A-6C show the knockdown efficiency of the androgen receptor via miR 3610. Wildtype mice were injected with 3e11 GC of AAV.PHP.eB.CB7.miR via tail vein. Brain and spinal cord were harvested at Day 14 and processed for RNA and protein analyses. FIG. 6A and FIG. 6B show androgen receptor mRNA expression levels in PBS- and miR 3610-treated brains. FIG. 6A (% control); FIG. 6B (fold expression). FIG. 6C shows androgen receptor protein levels in PBS- and miR 3610-treated brains.

FIGS. 7A-7E compare two miRNAs targeted against androgen receptor in brain and spinal cord. Adult male wild type mice (6-8 weeks old) received a single IV administration of AAV9.PHP.eB.CB7.CI.hARmiR3610.WPRE.rBG or AAV9.PHP.eB.CB7.CI.AR.miR3613.WPRE.rBG at a dose of 3.0×10¹¹ GC) (N=5/group). Additional wild type mice were administered vehicle (PBS) as a control (N=5). On Day 14, mice were necropsied. One hemisphere of the brain was collected to evaluate mouse AR mRNA expression (TaqMan qPCR). FIG. 7A shows androgen receptor mRNA expression levels in PBS-, miR 3610- or miR3613-treated brains. Fold change in expression for each animal was calculated based on the comparative Ct method and normalized to Gapdh. Error bars represent the standard deviation. FIG. 7B shows androgen receptor mRNA expression levels in PBS-, miR 3610- or miR3613-treated spinal cords. FIGS. 7C and 7D shows androgen receptor protein levels in miR NT- and miR 3610-treated brains (C) or miR3613-treated brains (D). FIG. 7E shows the quantification of androgen protein levels in percentage among all four groups.

FIG. 8 assesses promoter efficiency of CB7 and Syn. Wildtype mice were injected with 3e11 GC of the following vectors: AAV9-PHP.eB.CB7.CI.miR.NT.WPRE.rBG, AAV9.PHP.eB.CB7.CI.hARmiR3610.WPRE.rBG, AAV9-PHP.eB.Syn.PI.miR.NT.WPRE.bGH, or AAV9-PHP.eB.hSyn.PI.hARmiR3610.WPRE.bGH. Spinal cords were harvested at Day 14 and processed for RNA isolation and qPCR. The graph shows knockdown efficiency of the androgen receptor in the two promoters relative to their controls.

FIGS. 9A-9B show androgen receptor protein levels and survival in the AR97Q SBMA transgenic mice colony. In FIG. 9A spinal cords were harvested from transgenic mice and processed for Western blotting. The blot shows protein levels of the androgen receptor in AR97Q WT and HET male and female mice. FIG. 9B shows the survival plots for male and female AR97Q transgenic mice.

FIGS. 10A-10C show the effect of miR 3610 in AR97Q SBMA transgenic mice. 5 to 6 week old male transgenic mice were injected with 3e11 GC of AAV9.PHP.eB.CB7.CI.hARmiR3610.WPRE.rBG via tail vein or mice were left uninjected. Mice were followed for survival. The brains were harvested and processed for Western blotting. FIG. 10A shows androgen receptor protein levels in both groups. The age depicts when the brains were harvested post-injection. FIG. 10B shows protein quantification of both forms of the androgen receptor in treated mice relative to the uninjected group. FIG. 10C shows the survival plots for uninjected and treated mice.

FIGS. 11A-11C show the effect of miR 3610 in AR97Q SBMA transgenic mice. 3 week old male transgenic mice were injected with 3e11 GC of AAV9.PHP.eB.CB7.CI.hARmiR3610.WPRE.rBG via retro-orbital vein (ROV) or mice were left uninjected. Mice were followed for survival. The brains were harvested and processed for Western blotting. FIG. 11A shows the survival plots for the treated mice and AR97Q transgenic mice. FIG. 11B shows androgen receptor protein levels in both groups. The age depicts when the brains were harvested post-injection. FIG. 11C shows protein quantification of both forms of the androgen receptor in treated mice relative to the uninjected group.

FIGS. 12A-12I show the effect of miR 3610 in AR97Q SBMA neonatal transgenic mice. Neonatal transgenic mice of unknown sex and genotype were injected with 3e11 GC of AAVhu68.CB7.CI.hARmiR3610.WPRE.rBG (Group 2) via temporal vein or PBS (Group 1). Mice were followed for survival and genotypes/sex determined. The brains were harvested and processed for Western blotting. Male mice from each group were subjected to wire hang test at approximately 3 months of age. FIG. 12A shows androgen receptor protein levels in both groups. FIG. 12B and 12C show the survival plots for both groups for males (B) and females (C). FIG. 12D shows mouse AR expression in male WT SBMA mice spinal cord, western blot and quantification plot. FIG. 12E shows human and mouse AR expression in female het SBMA mice spinal cord, western blot and quantification plot. FIG. 12F shows mouse AR expression in female WT SBMA mice spinal cord, western blot and quantification plot. FIG. 12G and 12H show body weights for HET and WT mice given either PBS or AAVhu68.CB7.CI.hARmiR3610.WPRE.rBG for males (G) and females (H) over time. In FIG. 12I male mice from each group were subjected to wire hang test at approximately 3 months of age. The mouse was placed on top of the cage top, which is then inverted and placed over the home cage. The latency to when the mouse falls was recorded in seconds.

FIGS. 13A-13C demonstrate the effectiveness of miR 3610 in non-human primates (NHP). 5-yr old male rhesus macaque was injected ICM with 3e13 GC of AAVhu68.CB7.CI.hARmiR3610.WPRE.rBG. At day 35 the animal was sacrificed, and the spinal cord and liver were harvested. The spinal cord was processed for laser capture microdissection (LCM). Motor neurons were cut from the spinal cord sections and processed for qPCR. The liver was also processed for qPCR. Both spinal cord (A) and liver (B) display effective knockdown of the androgen receptor after treatment with miR 3610. FIG. 13C Rhesus macaque AR protein expression was also measured in liver samples (Western blotting) based on the percent expression relative to control animals. Expression was normalized to β-actin. Error bars represent the standard deviation

FIGS. 14A-14E show the results of the experiment described in Example 9. FIG. 14A shows a survival curve for PBS- and AAV.CB7.CI.AR.miR3610.WPRE.RBG-treated neonatal SBMA male transgenic mice (181.4 days). FIG. 14B shows a survival curve for AAVhu68.CB7.CI.AR.miR3610.WPRE.RBG-treated neonatal SBMA female transgenic mice (314 days). Mice performed wire hang behavioral assessments. Mice were placed on top of a wire grid, allowed to explore for 1 min. Then the grid was flipped over and the time to fall was recorded. Procedure was repeated 3 times. FIG. 14C, 14CD and 14E show latency to fall (seconds) for PBS- and vector-treated mice.

FIGS. 15A and 15B show the results of the experiment in Example 9, providing body weight data for heterozygous males (FIG. 15A) and females (FIG. 15B over the course of 56 weeks.

FIGS. 16A and 16B show results of laser-capture microdissection (LCM) of lumbar spinal cord motor neurons (MNs) following in vivo non-human primate injection of AAVhu68.CB7.CI.AR.miR3610.WPRE.rBG. Isolated MNs were RNA extracted and qPCR was performed with AR and rhGAPDH control primers. (FIG. 16A) qPCR was also performed on liver samples, however one of the animals (171299) had pre-existing NAbs that partially blocked expression in liver. FIG. 16B. FIG. 17-19C provides results from a study (V220121m) designed to study the efficacy of the test vector AAVhu68.CB7.CI.AR.miR3610.WPRE.rBG following intracerebroventricular (ICV) administration in transgenic mice (SBMA (AR97Q) mice to determine the minimum effective dose, over the course of 180 days. The low dose group (Group 1; received 3e9 GC (e=10^{-x})), Group 2 received 1e10 GC; Group 3 received 3e10, and a high dose group (Group 4) received 1e11 GC). Controls included vehicle (no vector; Group 5) and wild-type mice with vehicle (no vector; Group 6)

FIG. 17 provides body weight over the course of 24 weeks.

FIG. 18 provides a survival curve for the male transgenic mice. Group 1 shows a median survival of 120 days. Group 2 showed median survival of 117 days. Group 3 showed median survival of 168 days. Group 4 showed survival throughout the test period.

FIGS. 19A-19C show the results of the wire hang study for the animals on weeks 12, 14 and 16.

FIG. 20 shows the study design for the studying described in Example 13.

FIG. 21A and 22B show RNA levels in cervical motor neurons (FIG. 21A) and lumbar motor neurons (FIG. 21B).

### DETAILED DESCRIPTION OF THE INVENTION

Sequences, vectors and compositions are provided here for administering to a subject a nucleic acid sequence encoding at least one miRNA which specifically targets a site in the human androgen receptor gene or transcript of the subject. Novel miRNA sequences and constructs including the same are provided herein. These may be used alone or in combination with each other and/or other therapeutics for the treatment of SBMA.

As used herein the term “androgen receptor” refers to the androgen receptor (AR) gene which encodes the protein androgen receptor (AR) in humans [reproduced in SEQ ID NO: 6] (Uniprot P10275-1). Androgen receptor (AR), is a ligand-dependent nuclear transcription factor and member of the steroid hormone nuclear receptor family, and is expressed in a wide range of cells and tissues. The AR protein belongs to the class of nuclear receptors called activated class I steroid receptors, which also includes glucocorticoid receptor, progesterone receptor, and mineralocorticoid receptor. These receptors recognize canonical androgen response elements (AREs). The major domains of AR include N- and C-terminal activation domains, which are designated activation function-1 (AF-1) and AF-2, a ligand-binding domain, and a polyglutamine tract. This gene may alternatively be called: DIHYDROTESTOSTERONE RECEPTOR (DHTR); NUCLEAR RECEPTOR SUBFAMILY 3, GROUP C, MEMBER 4 (NR3C4). See, OMIM.org/entry/313700.

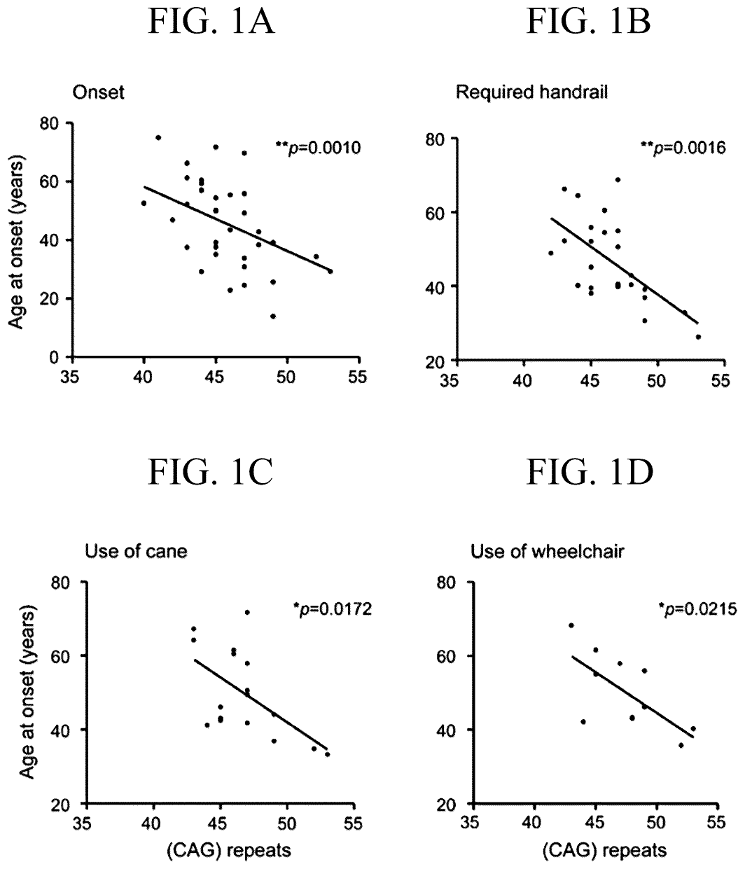

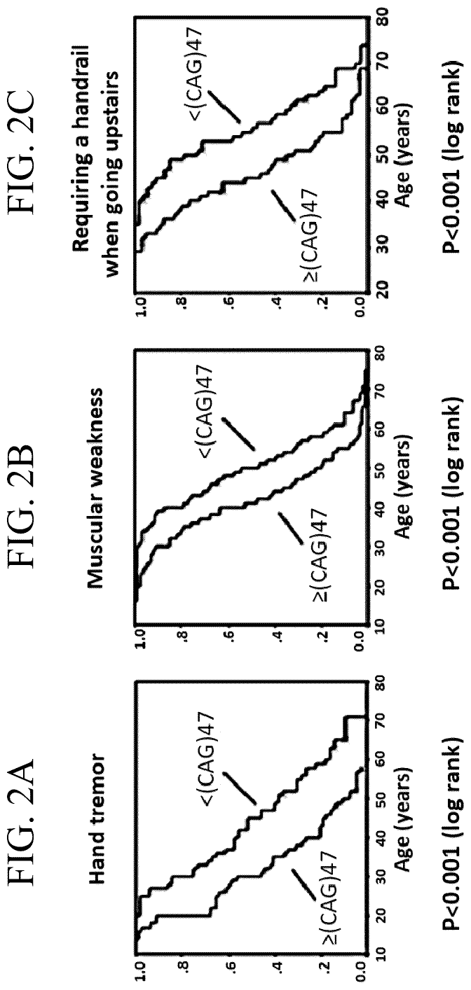

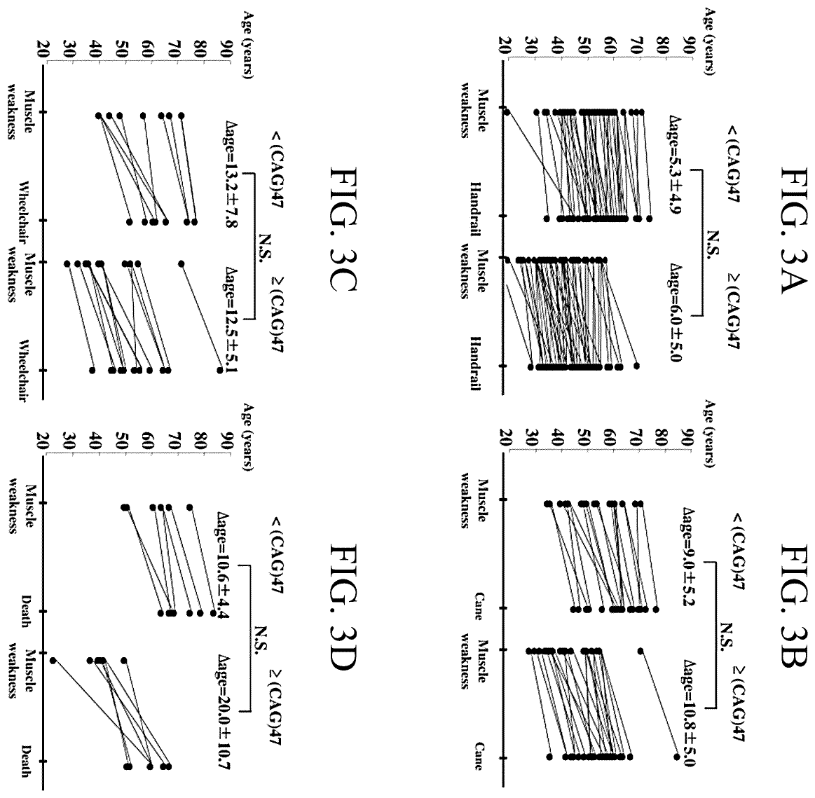

X-linked spinal and bulbar muscular atrophy (SBMA, SMAX1), also known as Kennedy disease, is caused by a trinucleotide CAG repeat expansion in exon 1 of the gene encoding the androgen receptor (AR; 313700.0014). CAG repeat numbers range from 38 to 62 in SBMA patients, whereas healthy individuals have 10 to 36 CAG repeats. SBMA onset has been shown to correlate with the number of CAG repeats (FIG. 1A-FIG. 1D; Fratta P, Nirmalananthan N, Masset L, et al. Correlation of clinical and molecular features in spinal bulbar muscular atrophy. Neurology. 2014;82(23):2077-2084. doi:10.1212/WNL.0000000000000507, which is incorporated herein by reference). However, the rate of progression is similar between all patients (FIG. 2A-FIG. 3D; Natural history of spinal and bulbar muscular atrophy (SBMA): a study of 223 Japanese patients; Brain. 2006;129(6):1446-1455, which is incorporated herein by reference.)

Described herein are vectors expressing artificial microRNAs (miRNAs) that repress expression of the endogenous androgen receptor. In a transgenic mouse model of SBMA, these vectors were shown to dramatically reduce expression of the mutant androgen receptor in spinal cord and improve motor function and survival.

As used herein, an “miRNA” refers to a microRNA, which is a small non-coding RNA molecule which regulates messenger RNA (mRNA) to inhibit protein translation. The miRNA is present in a pre-miRNA hairpin structure (also referred to as a stem-loop), which is eventually processed to the mature miRNA. The term “miRNA” and “miR” as used herein, can be used to refer to either unprocessed or mature miRNA (or sequences encoding the same). Generally, hairpin-forming RNAs have a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA have at least 1, at least 2, at least 3, at least 4, at least 5, at least 6, at least 7 at least 8, at least 9, or at least 10 base-pair mismatches.

Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue. As used herein, the miRNA contains a “seed sequence” which is a region of nucleotides which specifically binds to a target mRNA (e.g., in the human androgen receptor) by complementary base pairing, leading to destruction or silencing of the mRNA. Such silencing may result in downregulation rather than complete extinguishing of the endogenous hAR. The miRNA provided herein include a targeting sequence, which binds a target site on the mRNA of human androgen receptor. The targeting sequence comprises the seed sequence.

The encoded miRNA provided herein have been designed to specifically target the endogenous human androgen receptor gene in patients having SBMA. In certain embodiments the miRNA coding sequence comprises an anti-sense sequence in the following table 1.

TABLE 1

| | Target hAR | SEQ | miRNA antisense | SEQ |
|---|---|---|---|---|
| miR # | Sequence | ID NO | sequence | ID NO |
| 3610 | GAA CTA CAT | 1 | TCG AGT TCC | 2 |
| | CAA GGA ACT | | TTG ATG TAG | |
| | CGA | | TTC | |
| 3613 | CTA CAT CAA | 27 | CGA TCG AGT | 3 |
| | GGA ACT CGA | | TCC TTG ATG | |
| | TCG | | TAG | |

As used herein, an “miRNA target site”, “target sequence”, or “target region” is a sequence located on the DNA positive strand (5′ to 3′) (e.g., of hAR) and is at least partially complementary to a miRNA sequence, including the miRNA seed sequence (or targeting sequence). Typically, the miRNA target sequence is at least 7 nucleotides to about 28 nucleotides, at least 8 nucleotides to about 28 nucleotides, 7 nucleotides to 28 nucleotides, 8 nucleotides to 18 nucleotides, 12 nucleotides to 28 nucleotides, about 20 to about 26 nucleotides, about 18, 19, 20, 21, 22, 23, 24, 25, or 26 nucleotides, and contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA.

In certain embodiments the miRNA comprises a targeting sequence which binds the AR target site: GAA CTA CAT CAA GGA ACT CGA (SEQ ID NO: 1), or a sequence having 1, 2, 3, 4, or 5 substitutions therefrom (including truncations). In some embodiments, the targeting sequence is SEQ ID NO: 2. In other embodiments, the targeting sequence is SEQ ID NO: 3. In certain embodiments, the seed sequence is located on the mature miRNA (5′ to 3′) and generally starts at position 2 to 7, 2 to 8, or about 6 nucleotides from the 5′ end of the miRNA sense strand (from the 5′ end of the sense (+) strand) of the miRNA, although it may be longer in length. In certain embodiments, the length of the seed sequence is no less than about 30% of the length of the mature miRNA sequence, which may be at least 7 nucleotides to about 28 nucleotides, at least 8 nucleotides to about 28 nucleotides, 7 nucleotides to 28 nucleotides, 8 nucleotides to 18 nucleotides, 12 nucleotides to 28 nucleotides, about 20 to about 26 nucleotides, about 18, 19, 20, 21, 22, 23, 24, 25, or 26 nucleotides. In the examples provided herein, the miRNA is delivered in the form of a stem-loop miRNA precursor sequence, e.g., about 50 to about 80 nucleotides in length, or about 55 nucleotides to about 70 nucleotides, or 60 to 65 nucleotides in length. In some embodiments, the stem-loop miRNA precursor sequence is 64 nucleotides. In certain embodiments, this miRNA precursor comprises about 5 nucleotides, about a 21 nucleotide targeting sequence (which contains the seed sequence), about a 21 nucleotide stem loop and about a 20 nucleotide sense sequence, wherein the sense sequence corresponds to the anti-sense sequence with one, two, or three nucleotides being mismatched. In other embodiments, this miRNA precursor comprises about 5 nucleotides, about a 21 nucleotide targeting sequence, about a 21 nucleotide stem loop and about a 18 nucleotide sense sequence, wherein the sense sequence corresponds to the anti-sense sequence with one, two, or three nucleotides being mismatched. In certain embodiments, the miRNA targets the miRNA target site of SEQ ID NO: 1 or SEQ ID NO: 27, or a sequence having 1, 2, 3, 4, or 5 substitutions therefrom (including truncations) on human androgen receptor.

In one aspect, provided herein is an expression cassette comprising a nucleic acid sequence encoding at least one hairpin forming miRNA that comprises a targeting sequence which binds a miRNA target site on the mRNA of human androgen receptor, and inhibits expression of human androgen receptor. The coding sequence is operably linked to regulatory sequences which direct expression of the nucleic acid sequence in the subject. In some embodiments, the miRNA target site comprises: SEQ ID NO: 1, or a sequence having 1, 2, 3, 4, or 5 substitutions (or truncations) as compared to SEQ ID NO: 1. In some embodiments, the miRNA coding sequence comprises the sequence of TCG AGT TCC TTG ATG TAG TTC (SEQ ID NO: 2—3610 targeting sequence). In some embodiments, the miRNA target site comprises: SEQ ID NO: 27, or a sequence having 1, 2, 3, 4, or 5 substitutions (or truncations) as compared to SEQ ID NO: 27. In other embodiments, the miRNA coding sequence comprises the sequence of CGA TCG AGT TCC TTG ATG TAG (SEQ ID NO: 3—3613 targeting sequence). In some embodiments, the miRNA targeting sequence shares less than exact complementarity with the target site on the mRNA of human androgen receptor. In some embodiments, the miRNA coding sequence comprises the sequence of: a) TCG AGT TCC TTG ATG TAG TTC (SEQ ID NO: 2—3610) or a sequence having up to 10 substitutions; or b) CGA TCG AGT TCC TTG ATG TAG (SEQ ID NO: 3—3613), or a sequence having up to 10 substitutions. In another embodiment, the miRNA coding sequence comprises SEQ ID NO: 4, or a sequence having up to 30 substitutions. In yet another embodiment, the miRNA coding sequence comprises SEQ ID NO: 5, or a sequence having up to 30 substitutions.

An example of a suitable miRNA coding sequence is the sequence of SEQ ID NO: 4, which provides the coding sequence of a pre-miRNA hairpin, and includes the mature miR, miR3610. In certain embodiments, the miRNA coding sequence comprises SEQ ID NO: 4; a miRNA sequence comprising at least 60 consecutive nucleotides of SEQ ID NO: 4; or a miRNA sequence comprising at least 90% identity to SEQ ID NO: 4 which comprises a sequence with 100% identity to about nucleotide 6 to about nucleotide 26 of SEQ ID NO: 4. In still another embodiment, positions 6 to 26 of SEQ ID NO: 4 are retained, and an alternative sequence is selected for the stem-loop backbone. In another embodiment, the miRNA sequence comprises 5′ and/or 3′ flanking sequences. In certain embodiments, the miRNA sequence comprises SEQ ID NO: 11, or a miRNA sequence comprising at least 60 consecutive nucleotides of SEQ ID NO: 11; or a miRNA sequence comprising at least 90% identity to SEQ ID NO: 11.

Another example of a suitable miRNA coding sequence is the sequence of SEQ ID NO: 5, which provides the sequence encoding a pre-miRNA hairpin, and includes the mature miR, miR3613. In certain embodiments, the miRNA coding sequence comprises SEQ ID NO: 5; a miRNA sequence comprising at least 60 consecutive nucleotides of SEQ ID NO: 5; or a miRNA sequence comprising at least 90% identity to SEQ ID NO: 5 which comprises a sequence with 100% identity to about nucleotide 9 to about nucleotide 29 of SEQ ID NO: 5. In still another embodiment, positions 9 to 29 of SEQ ID NO: 5 are retained and an alternative sequence is selected for the stem-loop backbone. In another embodiment, the miRNA sequence comprises 5′ and/or 3′ flanking sequences. In certain embodiments, the miRNA sequence comprises SEQ ID NO: 12, or a miRNA sequence comprising at least 60 consecutive nucleotides of SEQ ID NO: 12; or a miRNA sequence comprising at least 90% identity to SEQ ID NO: 12.

In certain embodiments, an expression cassette is provided that includes SEQ ID NO: 26, or a sequence sharing at least 70%, 75%, 80%, 85%, 90%, 95%, 96%, 97%, 98%, 99%, or 99.9% identity therewith.

In certain embodiments, vector genome is provided that includes SEQ ID NO: 28, or a sequence sharing at least 70%, 75%, 80%, 85%, 90%, 95%, 96%, 97%, 98%, 99%, or 99.9% identity therewith.

In certain embodiments, the nucleic acid molecules (e.g., expression cassette or vector genome) may contain more than one miRNA coding sequence. Such nucleic acid molecule may comprise an miRNA encoding sequence having the sequence of one, two or more of: (a) an miRNA coding sequence comprising SEQ ID NO: 4; (b) an miRNA coding sequence comprising at least 60 consecutive nucleotides of SEQ ID NO: 4; (c) an miRNA coding sequence comprising at least 50% identity to SEQ ID NO: 4, which comprises a sequence with 100% identity to about nucleotide 6 to about nucleotide 26 of SEQ ID NO: 4; and/or (d) an miRNA coding sequence comprising TCG AGT TCC TTG ATG TAG TTC, SEQ ID NO: 2. In another embodiment, the nucleic acid molecule may comprise an miRNA coding sequence having the sequence of one, two or more of: (a) an miRNA coding sequence comprising SEQ ID NO: 5; (b) an miRNA coding sequence comprising at least 60 consecutive nucleotides of SEQ ID NO: 5; (c) an miRNA coding sequence comprising at least 50% identity to SEQ ID NO: 5, which comprises a sequence with 100% identity to about nucleotide 6 to about nucleotide 26 of SEQ ID NO: 5; and/or (d) an miRNA coding sequence comprising CGA TCG AGT TCC TTG ATG TAG, SEQ ID NO: 3.

As used herein, the terms “AAV.AR-miR” or “rAAV.AR.miR” are used to refer to a recombinant adeno-associated virus which has an AAV capsid having therewithin a vector genome comprising a nucleic acid sequence encoding at least one hairpin forming miRNA that comprises a targeting sequence that binds a miRNA target site on the mRNA of human androgen receptor, and inhibits expression of human androgen receptor, under the control of regulatory sequences. In some embodiments, the target sequence is that shown in SEQ ID NO: 1.

Specific capsid types may be specified, such as, e.g., AAV1.AR.miR, which refers to a recombinant AAV having an AAV1 capsid; AAVhu68.AR.miR, which refers to a recombinant AAV having an AAVhu68 capsid.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid. 5′ and 3′ ITR sequences are shown in SEQ ID NO: 7 and SEQ ID NO: 14, respectively. Alternate 5′ and 3′ ITR sequences are shown in SEQ ID NO: 34 and SEQ ID NO: 35, respectively.

Generally, an AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAVhu.68 capsid or variant thereof (see, e.g., WO 2018/160582 and U.S. Provisional Patent Application No. 63/093,275, filed Oct. 18, 2020, which are incorporated herein by reference). See, SEQ ID NO: 17. In another embodiment, the AAV capsid is an AAV.PHP.eb capsid (SEQ ID NO: 21). In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAVS, AAV6, AAV7, AAV8, AAV9, AAVrh10, AAVhu37, AAVrh32.33, AAV8bp, AAV7M8 and AAVAnc80, AAV1, AAV2, AAV3, AAV4, AAVS, AAV6, AAV7, AAV8, AAV9.47, AAV9(hu14), AAV10, AAV11, AAV12, AAVrh8, AAVrh74, AAV-DJ8, AAV-DJ, AAVhu68, without limitation. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), WO 2005/033321, WO 2018/160582 and U.S. Provisional Patent Application No. 63/093,275, filed Oct. 18, 2020 (AAVhu68), which are incorporated herein by reference. See, also WO 2019/168961 and WO 2019/169004, describing deamidation profiles for these and other AAV capsids. Other suitable AAVs may include, without limitation, AAVrh90 [PCT/US20/30273, filed Apr. 28, 2020], AAVrh91 [PCT/US20/30266, filed Apr. 28, 2020; U.S. Provisional Patent Application No. 63/065,616, filed Aug. 14, 2020], AAVrh92 [PCT/US20/30281, filed Apr. 28, 2020], AAVrh93 [PCT/US20/30281, filed Apr. 28, 2020], AAVrh91.93 [PCT/US20/30281, filed Apr. 28, 2020], which are incorporated by reference herein. Other suitable AAV include AAV3B variants which are described in U.S. Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, miRNA coding sequence, and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the miRNA. Suitable components of a vector genome are discussed in more detail herein.

In certain embodiments, a composition is provided which comprises an aqueous liquid suitable for intrathecal injection and a stock of vector (e.g., rAAV) having a AAV capsid which preferentially targets cells in the central nervous system and/or the dorsal root ganglia (e.g., CNS), including, e.g., nerve cells (such as, pyramidal, purkinje, granule, spindle, and interneuron cells) and glia cells (such as astrocytes, oligodendrocytes, microglia, and ependymal cells), wherein the vector has at least one miRNA specific for AR for delivery to the central nervous system (CNS). In certain embodiments, the composition comprising one or more vectors as described herein is formulated for sub-occipital injection into the cisterna magna (intra-cisterna magna). In certain embodiments, the composition is administered via a computed tomography-(CT-) rAAV injection. In certain embodiments, the patient is administered a single dose of the composition.

As used herein, an “expression cassette” refers to a nucleic acid polymer which comprises the miRNA coding sequences targeting human AR, promoter, and may include other regulatory sequences therefor, which cassette may be packaged into a vector (e.g., rAAV, lentivirus, retrovirus, etc).

### rAAV

Recombinant parvoviruses are particularly well suited as vectors for treatment of SBMA. As described herein, recombinant parvoviruses may contain an AAV capsid (or bocavirus capsid). In certain embodiments, the capsid targets cells within the dorsal root ganglion and/or cells within the lower motor neurons and/or primary sensory neurons. In certain embodiments, compositions provided herein may have a single rAAV stock which comprises an rAAV comprising a miRNA specifically targeting hAR in order to downregulate the endogenous hAR levels.

For example, vectors generated using AAV capsids from Clade F (e.g., AAVhu68 or AAV9) can be used to produce vectors which target and express miRs in the CNS. Alternatively, vectors generated using AAV capsids from Clade A (e.g., AAV1, AAVrh91) may be selected. In still other embodiments, other parvovirus or other AAV viruses may be suitable sources of AAV capsids.

An AAV1 capsid refers to a capsid having AAV vp1 proteins, AAV vp2 proteins and AAV vp3 proteins. In particular embodiments, the AAV1 capsid comprises a pre-determined ratio of AAV vp1 proteins, AAV vp2 proteins and AAV vp3 proteins of about 1:1:10 assembled into a T1 icosahedron capsid of 60 total vp proteins. An AAV1 capsid is capable of packaging genomic sequences to form an AAV particle (e.g., a recombinant AAV where the genome is a vector genome). Typically, the capsid nucleic acid sequences encoding the longest of the vp proteins, i.e., VP1, is expressed in trans during production of an rAAV having an AAV1 capsid are described in, e.g., U.S. Pat. Nos. 6,759,237, 7,105,345, 7,186,552, 8,637,255, and 9,567,607, which are incorporated herein by reference. See, also, WO 2018/168961, which is incorporated by reference. In certain embodiments, AAV1 is characterized by a capsid composition of a heterogeneous population of VP isoforms which are deamidated as defined in the following table, based on the total amount of VP proteins in the capsid, as determined using mass spectrometry. In certain embodiments, the AAV capsid is modified at one or more of the following positions, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, an AAV1 mutant is constructed in which the glycine following the N at position 57, 383, 512 and/or 718 are preserved (i.e., remain unmodified). In certain embodiments, the NG at the four positions identified in the preceding sentence are preserved with the native sequence. In certain embodiments, an artificial NG is introduced into a different position than one of the positions identified in the table above.

As used herein, an AAVhu68 capsid refers to a capsid as defined in WO 2018/160582, incorporated herein by reference. See, SEQ ID NO: 17. A production sequence for AAVhu68 can be found in SEQ ID NO: 16 and in SEQ ID NO: 18 (capsid only coding sequence).

The rAAVhu68 resulting from production using a single vp1 nucleic acid sequence produces heterogeneous populations of vp1 proteins, vp2 proteins and vp3 proteins. These subpopulations include, at a minimum, deamidated asparagine (N or Asn) residues. For example, asparagines in asparagine—glycine pairs are highly deamidated. In certain embodiments, the vp2 and/or vp3 proteins may be expressed additionally or alternatively from different nucleic acid sequences than the vp1, e.g., to alter the ratio of the vp proteins in a selected expression system.

In certain embodiments, the AAVhu68 capsid comprises AAVhu68 VP1, VP2 and VP3 proteins which are, respectively, amino acids 1-736, amino acids 138-736, and amino acids 203-736 of SEQ NO: 17, and/or variants thereof, wherein said variants are said AAVhu68 VP1, VP2 and VP3 proteins but with (i) one or more modifications selected from: acetylated lysine, phosphorylates serine and/or threonine, isomerized aspartic acid, oxidized tryptophan and/or methionine, or an amidated amino acid; and/or (ii) deamidation of N57, N66, N94, N113, N252, N253, Q259, N270, N303, N304, N305, N314, N319, N328, N329, N336, N409, N452, N477, N512, N515, N598, Q599, N628, N651, N663, N709, N735 or a combination thereof, as determined using a suitable method (e.g., mass spectrometry).

In certain embodiments, the AAVhu68 capsid comprises a heterogenous population of AAVhu68 vp1 proteins selected from: vp1 proteins produced by expression from a nucleic acid sequence which encodes the predicted amino acid sequence of 1 to 736 of SEQ ID NO: 17, vp1 proteins produced from SEQ ID NO: 18, or vp1 proteins produced from a nucleic acid sequence at least 70%, 75%, 80%, 85%, 90%, 91%, 92%, 93%, 94%, 95%, 96%, 97%, 98%, or 99% identical to SEQ ID NO: 18 which encodes the predicted amino acid sequence of 1 to 736 of SEQ ID NO: 17, a heterogenous population of AAVhu68 vp2 proteins selected from: vp2 proteins produced by expression from a nucleic acid sequence which encodes the predicted amino acid sequence of at least about amino acids 138 to 736 of SEQ ID NO: 17, vp2 proteins produced from a sequence comprising at least nucleotides 412 to 2211 of SEQ ID NO: 18, or vp2 proteins produced from a nucleic acid sequence at least 70%, 75%, 80%, 85%, 90%, 91%, 92%, 93%, 94%, 95%, 96%, 97%, 98%, or 99% identical to at least nucleotides 412 to 2211 of SEQ ID NO: 18 which encodes the predicted amino acid sequence of at least about amino acids 138 to 736 of SEQ ID NO: 17, and a heterogenous population of AAVhu68 vp3 proteins selected from: vp3 produced by expression from a nucleic acid sequence which encodes the predicted amino acid sequence of at least about amino acids 203 to 736 of SEQ ID NO: 17, vp3 proteins produced from a sequence comprising at least nucleotides 607 to 2211 of SEQ ID NO: 18, or vp3 proteins produced from a nucleic acid sequence at least 70%, 75%, 80%, 85%, 90%, 91%, 92%, 93%, 94%, 95%, 96%, 97%, 98%, or 99% identical to at least nucleotides 607 to 2211 of SEQ ID NO: 18 which encodes the predicted amino acid sequence of at least about amino acids 203 to 736 of SEQ ID NO: 17.

In certain embodiments, AAVhu68 capsid comprises (a) AAVhu68 VP1, AAVhu68 VP2 and AAVhu68 VP3 proteins produced by expression from a nucleic acid sequence which encodes the amino acid sequence of 1 to 736 of SEQ ID NO: 17; and/or (b) AAVhu68 VP1, AAVhu68 VP2 and AAVhu68 VP3 proteins which are, respectively, amino acids 1 to 736, amino acids 138 to 736, and amino acids 203 to 736 of SEQ ID NO: 17, which further comprise at least 60% deamidation of the asparagines at positions 57, 329, 452 and 512 of SEQ ID NO: 17 as determined using mass spectrometry. In certain embodiments, deamidation is at least 80%, at least 90%, at least 95%, or 100% at positions 57, 329, 452 and 512 of SEQ ID NO: 17, as determined using mass spectrometry. The AAVhu68capsids may include other post-translational modifications, including deamidation at other positions, while retaining glutamic acid at position 67 and valine at position 157.

In certain embodiments, the AAVhu68 capsid is produced using an engineered AAVhu68 coding sequence. See, e.g., U.S. Provisional Patent Application No. 63/093,275, filed Oct. 18, 2020, and International Patent Application No. PCT/US21/55436, filed Oct. 18, 2021, each of which is incorporated herein by reference. The capsid may be produced in any suitable production cell system, including cell culture, adherent cells, or a cell suspension.

Genomic sequences which are packaged into an AAV capsid and delivered to a host cell are typically composed of, at a minimum, a transgene (e.g., miRNA) and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 130 or 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. A shortened version of the 5′ ITR, termed AITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the vector (e.g., an rAAV), the vector also includes conventional control elements necessary which are operably linked to the transgene in a manner which permits its transcription, translation and/or expression in a cell. As used herein, the term “expression” or “gene expression” refers to the process by which information from a gene is used in the synthesis of a functional gene product. The gene product may be a miRNA, a protein, a peptide, or a nucleic acid polymer (such as a RNA, a DNA or a PNA).

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. In certain embodiments, the promoter is a chicken beta actin promoter with CMV enhancer elements, e.g., the CB7 promoter (SEQ ID NO: 23). In another embodiment, the CB7 promoter has the sequence of SEQ ID NO: 24. In certain embodiments, the CB7 promoter includes a CMV enhancer (SEQ ID NO: 8 or SEQ ID NO: 29) and a chicken beta-actin promoter (SEQ ID NO: 9 or SEQ ID NO: 30); the expression cassette may also contain a chimeric intron (SEQ ID NO: 10 or SEQ ID NO: 31). In particular embodiments, a tissue specific promoter for the central nervous system is selected. For example, the promoter may be a neural cell promoter, e.g., gfaABC(1)D promoter (Addgene #50473)), or the human Syn promoter (the sequence is available from Addgene, Ref #50465; SEQ ID NO: 15).

Other suitable promoters include, e.g., constitutive promoters, regulatable promoters [see, e.g., WO 2011/126808 and WO 2013/04943], or a promoter responsive to physiologic cues. The promoter can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, melanin-concentrating hormone (MCH) promoter, CBA, matrix metalloprotein promoter (MPP), and the chicken beta-actin promoter.

In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., rabbit beta globin (Seq ID NO: 25), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., M A Zanta-Boussif, et al, Gene Therapy (2009) 16: 605-619. An example of a suitable WPRE is shown in SEQ ID NO: 13 or SEQ ID NO: 32.

In one embodiment, the vector genome comprises: an AAV 5′ ITR, a promoter, an optional enhancer, an optional intron, a coding sequence for a miRNA which targets human androgen receptor, a poly A, and an AAV 3′ ITR. In certain embodiments, the vector genome comprises: a AAV 5′ ITR, a promoter, an optional enhancer, an optional intron, a coding sequence for a miRNA which targets human androgen receptor, an optional WPRE, a poly A, and an AAV 3′ ITR. In certain embodiments, the vector genome comprises: a AAV 5′ ITR, a promoter, an enhancer, an intron, a coding sequence for a miRNA which targets human androgen receptor sequence of SEQ ID NO: 1, a WPRE, a poly A, and an AAV 3′ ITR. In certain embodiments, the vector genome comprises: a AAV 5′ ITR, a CB7 promoter/enhancer, a chicken-beta intron, a coding sequence for a miRNA which targets human androgen receptor sequence of SEQ ID NO: 1, a WPRE, a rabbit beta globin poly A, and an AAV 3′ ITR. In certain embodiments, the vector genome comprises: a AAV 5′ ITR, a CB7 promoter/enhancer, a chicken-beta intron, a coding sequence for a miRNA which targets the human androgen receptor sequence of SEQ ID NO: 1 which comprises SEQ ID NO: 2, or a sequence having up to 10 substitutions therefrom, a WPRE, a rabbit beta globin poly A, and an AAV 3′ ITR. In certain embodiments, the vector genome comprises: a AAV 5′ ITR, a CB7 promoter/enhancer, a chicken-beta intron, a coding sequence for a miRNA which targets the human androgen receptor sequence of SEQ ID NO: 27 which comprises SEQ ID NO: 3, or a sequence having up to 10 substitutions therefrom, a WPRE, a rabbit beta globin poly A, and an AAV 3′ ITR. The miRNA coding sequences are selected from those defined in the present specification. Other elements of the vector genome or variations on these sequences may be selected for the vector genomes for certain embodiments of this invention.

### Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In certain embodiments, the production plasmid comprises a vector genome for packaging into a capsid which comprises at least one miRNA sequence specific for human androgen receptor in a SBMA patient, operably linked to regulatory sequences which direct expression of the miRNA in the patient.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the miRNA construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding the transgene (e.g., miRNA) operably linked to sequences which direct expression of the transgene in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Typically, the rep functions are from the same AAV source as the AAV providing the ITRs flanking the vector genome. In the examples herein, the AAV2 ITRs are selected and the AAV2 rep is used. Optionally, other rep sequences or another rep source (and optionally another ITR source) may be selected. For example, the rep may be, but is not limited to, AAV1 rep protein, AAV2 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293). Methods for manufacturing the therapeutic vectors described herein include methods well known in the art such as generation of plasmid DNA used for production of the therapeutic vectors, generation of the vectors, and purification of the vectors. In some embodiments, the therapeutic vector is an AAV vector and the plasmids generated are an AAV cis-plasmid encoding the AAV genome and the gene of interest (e.g., miRNA), an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media.

In certain embodiments, the manufacturing process for rAAV.AR-miR involves transient transfection of HEK293 cells with plasmid DNA. A single batch or multiple batches are produced by PEI-mediated triple transfection of HEK293 cells in PALL iCELLis bioreactors. Harvested AAV material are purified sequentially by clarification, TFF, affinity chromatography, and anion exchange chromatography in disposable, closed bioprocessing systems where possible.

The harvested vector-containing cells and culture media are referred to herein as crude cell harvest. In yet another system, the therapeutic vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, which are incorporated herein by reference.

The crude cell harvest may thereafter be subject to additional method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, which is incorporated by reference herein. Purification methods for AAV8, International Patent Application No. PCT/US2016/065976, filed Dec. 9, 2016, and rh10, International Patent Application No. PCT/US16/66013, filed Dec. 9, 2016, entitled “Scalable Purification Method for AAVrh10”, also filed Dec. 11, 2015, and for AAV1, International Patent Application No. PCT/US2016/065974, filed Dec. 9, 2016, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL—GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April;25(2):115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1), which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### NON-AAV AND NON-VIRAL VECTORS

A “vector” as used herein is a biological or chemical moiety comprising a nucleic acid sequence which can be introduced into an appropriate target cell for replication or expression of said nucleic acid sequence. Examples of vectors include, but are not limited to recombinant viruses, a plasmid, lipoplexes, polymersomes, polyplexes, dendrimers, cell penetrating peptide (CPP) conjugates, magnetic particles, or nanoparticles. In one embodiment, a vector is a nucleic acid molecule into which an exogenous or heterologous or engineered miRNA may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In one embodiment, the vector is a non-viral plasmid that comprises an expression cassette described thereof, e.g., “naked DNA”, “naked plasmid DNA”, RNA, mRNA, shRNA, RNAi, etc. Optionally the plasmid or other nucleic acid sequence is delivered via a suitable device, e.g., via electrospray, electroporation. In other embodiments, the nucleic acid molecule is coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., WO2014/089486, US 2018/0353616A1, US2013/0037977A1, WO2015/074085A1, U.S. Pat. Nos. 9,670,152B2, and 8,853,377B2, X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiment, a non-viral vector is used for delivery of a miRNA transcript targeting endogenous hAR, e.g., at SEQ ID NO: 1 or SEQ ID NO: 27. In some embodiments, the miRNA is delivered at an amount greater than about 0.5 mg/kg (e.g., greater than about 1.0 mg/kg, 1.5 mg/kg, 2.0 mg/kg, 2.5 mg/kg, 3.0 mg/kg, 4.0 mg/kg, 5.0 mg/kg, 6.0 mg/kg, 7.0 mg/kg, 8.0 mg/kg, 9.0 mg/kg, or 10.0 mg/kg) body weight of miRNA per dose. In some embodiments, the miRNA is delivered at an amount ranging from about 0.1-100 mg/kg (e.g., about 0.1-90 mg/kg, 0.1-80 mg/kg, 0.1-70 mg/kg, 0.1-60 mg/kg, 0.1-50 mg/kg, 0.1-40 mg/kg, 0.1-30 mg/kg, 0.1-20 mg/kg, 0.1-10 mg/kg) body weight of miRNA per dose. In some embodiments, the miRNA is delivered at an amount of or greater than about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg per dose.

In certain embodiments, miRNA transcripts are encapsulated in a lipid nanoparticle (LNP). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more miRNA to one or more target cells (e.g., dorsal root ganglion, lower motor neurons and/or upper motor neurons, or the cell types identified above in the CNS). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a miRNA to a target cell. Useful lipid nanoparticles for miRNA comprise a cationic lipid to encapsulate and/or enhance the delivery of miRNA into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27(4):710-728). In some embodiments, LNP comprises a cationic lipids (i.e. N-11-(2,3-dioleoyloxy)propyll-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). See, e.g., WO2014/089486, US 2018/0353616A1, US2013/0037977A1, WO2015/074085A1, U.S. Pat. Nos. 9,670,152B2, and 8,853,377B2, which are incorporated by reference.

In certain embodiments, the vector described herein is a “replication-defective virus” or a “viral vector” which refers to a synthetic or artificial viral particle in which an expression cassette containing a nucleic acid sequence encoding at least one miRNA targeting hAR. Replication-defective viruses cannot generate progeny virions but retain the ability to infect target cells. In one embodiment, the genome of the viral vector does not include genes encoding the enzymes required to replicate (the genome can be engineered to be “gutless”—containing only the nucleic acid sequence encoding E2 flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant viral vector may be any suitable replication-defective viral vector, including, e.g., a recombinant adeno-associated virus (AAV), an adenovirus, a bocavirus, a hybrid AAV/bocavirus, a herpes simplex virus or a lentivirus.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the miRNA is desired. In certain embodiments, the term “target cell” is intended to reference the cells of the subject being treated for SBMA. Examples of target cells may include, but are not limited to, cells within the central nervous system.

### Compositions

Provided herein are compositions containing at least one vector comprising a sequence encoding an miRNA targeting human androgen receptor (e.g., an rAAV.AR-miR stock) and/or at least one vector comprising AR-miR and/or at least one vector comprising AR-miR stock, and an optional carrier, excipient and/or preservative. A vector (e.g., rAAV) stock refers to a plurality of vectors which are the same, e.g., such as in the amounts described below in the discussion of concentrations and dosage units.

In certain embodiments, a composition comprises at least a virus stock which is a recombinant AAV (rAAV) suitable for use in treating SBMA alone or in combination with other vector stock(s) or composition(s). In certain embodiments, a composition comprises a virus stock which is a recombinant AAV (rAAV) suitable for use in treating SBMA, said rAAV comprising: (a) an adeno-associated virus capsid, and (b) a vector genome packaged in the AAV capsid, said vector genome comprising AAV inverted terminal repeats, a coding sequence for at least one miRNA specifically targeted to human androgen receptor, and regulatory sequences which direct expression of the miRNA. In certain embodiments, the vector genome comprises a promoter, an enhancer, an intron, a miRNA coding sequence targeting the hAR sequence of SEQ ID NO: 1, a WPRE, and a polyadenylation signal. In certain embodiments, the vector genome further comprises an AAV2 5′ ITR and an AAV2 3′ ITR which flank all elements of the vector genome. In certain embodiments, the vector genome comprises a promoter, an enhancer, an intron, a miRNA coding sequence encoding miR 3610, a WPRE, and a polyadenylation signal. In certain embodiments, the vector genome comprises a promoter, an enhancer, an intron, a miRNA coding sequence encoding miR 3613, a WPRE, and a polyadenylation signal.

The rAAV.AR-miR may be suspended in a physiologically compatible carrier to be administered to a human SBMA patient. In certain embodiments, for administration to a human patient, the vector is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, or a pH of 7.2 to 7.4, for intrathecal delivery, a pH within this range may be desired; whereas for intravenous delivery, a pH of about 6.8 to about 7.2 may be desired. However, other pHs within the broadest ranges and these subranges may be selected for other route of delivery.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2 or a pH of 7.4.

In another embodiment, the formulation may contain a buffered saline aqueous solution comprising 1 mM Sodium Phosphate (Na3PO4), 150 mM sodium chloride (NaCl), 3mM potassium chloride (KCl), 1.4 mM calcium chloride (CaCl2), 0.8 mM magnesium chloride (MgCl2), and 0.001% Kolliphor® 188. See, e.g., harvardapparatus.com/harvard-apparatus-perfusion-fluid.html. In certain embodiments, Harvard's buffer is preferred.

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

Optionally, the compositions may contain, in addition to the vector (e.g., rAAV) and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Optionally, routes other than intrathecal administration may be used, such as, e.g., direct delivery to a desired organ (e.g., the liver (optionally via the hepatic artery), lung, heart, eye, kidney), oral, inhalation, intranasal, intratracheal, intraarterial, intraocular, intravenous, intramuscular, subcutaneous, intradermal, and other parental routes of administration. Routes of administration may be combined, if desired.

Dosages of the vector will depend primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In certain embodiments, the dose is in the range of about 1×10⁹ GC/g brain mass to about 1×10¹² GC/g brain mass. In certain embodiments, the dose is in the range of about 1×10¹⁰ GC/g brain mass to about 3.33×10¹¹ GC/g brain mass. In certain embodiments, the dose is in the range of about 3.33×10¹¹ GC/g brain mass to about 1.1×10¹² GC/g brain mass. In certain embodiments, the dose is in the range of about 1.1×10¹² GC/g brain mass to about 3.33×10¹³ GC/g brain mass. In certain embodiments, the dose is lower than 3.33×10¹¹ GC/g brain mass. In certain embodiments, the dose is lower than 1.1×10¹² GC/g brain mass. In certain embodiments, the dose is lower than 3.33×10¹³ GC/g brain mass. In certain embodiments, the dose is about 1×10¹⁰ GC/g brain mass. In certain embodiments, the dose is about 2×10¹⁰ GC/g brain mass. In certain embodiments, the dose is about 2×10¹⁰ GC/g brain mass. In certain embodiments, the dose is about 3×10¹⁰ GC/g brain mass. In certain embodiments, the dose is about 4×10¹⁰ GC/g brain mass. In certain embodiments, the dose is about 5×10¹⁰ GC/g brain mass. In certain embodiments, the dose about 6×10¹⁰ GC/g brain mass. In certain embodiments, the dose is about 7×10¹⁰ GC/g brain mass. In certain embodiments, the dose about 8×10¹⁰ GC/g brain mass. In certain embodiments, the dose is about 9×10¹⁰ GC/g brain mass. In certain embodiments, the dose is about 1×10¹¹ GC/g brain mass. In certain embodiments, the dose is about 2×10¹¹ GC/g brain mass. In certain embodiments, the dose is about 3×10¹¹ GC/g brain mass. In certain embodiments, the dose is about 4×10¹¹ GC/g brain mass. In certain embodiments, the dose is administered to humans as a flat dose in the range of about 1.44×10¹³ to 4.33×10¹⁴ GC of the rAAV. In certain embodiments, the dose is administered to humans as a flat dose in the range of about 1.44×10¹³ to 2×10¹⁴ GC of the rAAV. In certain embodiments, the dose is administered to humans as a flat dose in the range of about 3×10¹³ to 1×10¹⁴ GC of the rAAV. In certain embodiments, the dose is administered to humans as a flat dose in the range of about 5×10¹³ to 1×10¹⁴ GC of the rAAV. In some embodiments, the compositions can be formulated in dosage units to contain an amount of AAV that is in the range of about 1×10¹³ to 8×10¹⁴ GC of the rAAV. In some embodiments, the compositions can be formulated in dosage units to contain an amount of rAAV that is in the range of about 1.44×10¹³ to 4.33×10¹⁴ GC of the rAAV. In some embodiments, the compositions can be formulated in dosage units to contain an amount of rAAV that is in the range of about 3×10¹³ to 1×10¹⁴ GC of the rAAV. In some embodiments, the compositions can be formulated in dosage units to contain an amount of rAAV that is in the range of about 5×10¹³ to 1×10¹⁴ GC of the rAAV.

In certain embodiments, the vector is administered to a subject in a single dose. In certain embodiments, vector may be delivered via multiple injections (for example 2 doses) is desired.

The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions provided herein.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna. In certain embodiments, delivery is accomplished through the use of a subdurally implantable device, such as an Ommaya reservoir.

As used herein, the terms “intracisternal delivery” or “intracisternal administration” refer to a route of administration directly into the cerebrospinal fluid of the cisterna magna cerebellomedularis, more specifically via a suboccipital puncture or by direct injection into the cisterna magna or via permanently positioned tube.

Compositions comprising the miR target sequences described herein for repressing endogenous hAR (e.g., in SBMA patients) are generally targeted to one or more different cell types within the central nervous system, including, but not limited to, neurons (including, e.g., lower motor neurons and/or primary sensory neurons. These may include, e.g., pyramidal, purkinje, granule, spindle, and interneuron cells).

### Uses

The vectors and compositions provided herein are useful for treating a patient having Spinal and Bulbar Muscular Atrophy (SBMA) or various symptoms associated therewith. A regimen for treating a patient having SBMA is provided. In certain embodiments, this regimen comprises administering a recombinant nucleic acid sequence encoding at least one hairpin forming miRNA that comprises a targeting sequence that binds a miRNA target site on the mRNA of human androgen receptor, operably linked to regulatory sequences which direct expression of the nucleic acid sequence in the subject, wherein the miRNA inhibits expression of human androgen receptor. In certain embodiments, the miRNA target site comprises: GAA CTA CAT CAA GGA ACT CGA (SEQ ID NO: 1). In certain embodiments, an AAV, expression cassette, nucleic acid, or composition as described herein are used.

In certain embodiments, the composition is formulated to be administered intrathecally at a dose of 1×10¹⁰ GC/g brain mass to 3.33×10¹¹ GC/g brain mass of the rAAV. In other embodiments, the patient is a human adult and is administered a dose of 1.44×10¹³ to 4.33×10¹⁴ GC of the rAAV. In other embodiments, the composition is delivered intrathecally, via intracerebroventricular delivery, or via intraparenchymal delivery. In other embodiments, the composition is administered as a single dose via a computed tomography-(CT-) guided sub-occipital injection into the cisterna magna (intra-cisterna magna) (ICM).

Optionally, the vectors and compositions provided herein may be used in combination with one or more co-therapies selected from: acetaminophen, nonsteroidal anti-inflammatory drugs (NSAIDs), tricyclic antidepressants or antiepileptic drugs, such as carbamazepine or gabapentin. Other co-therapies include, a pegylated IGF-1 mimetic (e.g., BVS857) (see, e.g., Grunseich C, et al, BVS857 study group. Safety, tolerability, and preliminary efficacy of an IGF-1 mimetic in patients with spinal and bulbar muscular atrophy: a randomised, placebo-controlled trial. Lancet Neurol. 2018 December;17(12):1043-1052. doi: 10.1016/S1474-4422(18)30320-X. Epub 2018 Oct. 15. PMID: 30337273), an antisense oligonucleotide that suppresses AR gene expression (see, e.g., Cell Rep. 2014 May 8; 7(3): 774-784. doi:10.1016/j.celrep.2014.02.008), intrabody (e.g., INT41), a small-molecule Nrf1 or Nrf2 activator (e.g., AJ201, ALZ002 aka ASC-JM17) (see, e.g., Human Molecular Genetics, 2016, Vol. 25, No. 10), leuprorelin acetate (see, e.g., Lancet Neurol 2010; 9: 875-84), dutasteride (synthetic 4-azasteroid compound) (see, e.g., 1 Lancet Neurol. 2011 February; 10(2): 140-147. doi:10.1016/51474-4422(10)70321-5), mrR-196a, Src kinase inhibitor (e.g., A419259), AR isoform 45 (e.g., AAV9-AR45), and clenbuterol (see e.g., Querin G, D'Ascenzo C, Peterle E, et al. Pilot trial of clenbuterol in spinal and bulbar muscular atrophy. Neurology 2013;80:2095-8). In still other embodiments, the vectors may be delivered in a combination with an immunomodulatory regimen involving one or more steroids, e.g., prednisone.

As used herein, the term Computed Tomography (CT) refers to radiography in which a three-dimensional image of a body structure is constructed by computer from a series of plane cross-sectional images made along an axis.

A “self-complementary nucleic acid” refers to a nucleic acid capable of hybridizing with itself (i.e., folding back upon itself) to form a single-stranded duplex structure, due to the complementarity (e.g., base-pairing) of the nucleotides within the nucleic acid strand. Self-complementary nucleic acids can form a variety of secondary structures, such as hairpin loops, loops, bulges, junctions and internal bulges. Certain self-complementary nucleic acids (e.g., miRNA or AmiRNA (artificial miRNA)) perform regulatory functions, such as gene silencing.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

As used herein, “disease”, “disorder” and “condition” are used interchangeably, to indicate an abnormal state in a subject.

As used herein, the term “SBMA-related symptom(s)” or “symptom(s)” refers to symptom(s) found in SBMA patients as well as in SBMA animal models. Early symptoms of SBMA may include one or more of weakness/cramps in arm and leg muscles, face, mouth, and tongue muscle weakness, difficulty with speaking and swallowing, twitching (Fasciculations), tremors and trembling in certain positions, enlarged breasts, (gynecomastia), numbness, infertility, and testicular atrophy. The disease affects the lower motor neurons that are responsible for the movement of many muscles in the legs, arms, mouth, and throat. Affected individuals will show signs of twitching, often in the tongue and/or hand, followed by muscle weakness and problems with facial muscles. These neurons, which connect the spinal cord to the muscles, become defective and die, so the muscles cannot contract. The destruction of these nerves is the main reason for the numbness, muscle weakness, and inability to control muscle contraction. With lack of normal neuromuscular function, a patient may experience hypertrophied calves in which the calf muscles thicken due to muscle cramps. In some cases, patients may also have one side of the body more affected than the other side.

The disease also affects nerves that control the bulbar muscles, which are important for breathing, speaking, and swallowing. Androgen insensitivity can also occur, sometimes beginning in adolescence and continuing through adulthood, characterized by enlarged breasts, decreased masculine appearance, and infertility. Patients may experience problems such as low sperm count and erectile dysfunction.

“Patient” or “subject” as used herein means a male or female human, dogs, and animal models used for clinical research. In one embodiment, the subject of these methods and compositions is a human diagnosed with SBMA. In certain embodiments, the human subject of these methods and compositions is a prenatal, a newborn, an infant, a toddler, a preschool, a grade-schooler, a teen, a young adult or an adult. In a further embodiment, the subject of these methods and compositions is an adult SBMA patient. In a further embodiment, the subject is a male.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, an “expression cassette” refers to a nucleic acid molecule which comprises a coding sequence, promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the coding sequence for the miRNA described herein flanked by packaging signals of the viral genome and other expression control sequences such as those described herein.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

The term “translation” in the context of the present invention relates to a process at the ribosome, wherein an mRNA strand controls the assembly of an amino acid sequence to generate a protein or a peptide.

### Specific Embodiments

1. An expression cassette comprising a nucleic acid sequence encoding at least one hairpin forming miRNA that comprises a targeting sequence that binds a miRNA target site on the mRNA of human androgen receptor, operably linked to regulatory sequences which direct expression of the nucleic acid sequence in the subject, wherein the miRNA inhibits expression of human androgen receptor.
2. The expression cassette of embodiment 1, wherein the miRNA target site comprises: GAA CTA CAT CAA GGA ACT CGA (SEQ ID NO: 1).
3. The expression cassette of embodiment 1 or embodiment 2, wherein the miRNA coding sequence comprises the sequence of TCG AGT TCC TTG ATG TAG TTC (SEQ ID NO: 2).
4. The expression cassette of embodiment 1 or embodiment 2, wherein the miRNA coding sequence comprises the sequence of CGA TCG AGT TCC TTG ATG TAG (SEQ ID NO: 3).
5. The expression cassette of any one of embodiments 1 to 4, wherein, the miRNA targeting sequence shares less than exact complementarity with the target site on the mRNA of human androgen receptor.
6. The expression cassette of any one of embodiments 1 to 5, wherein the miRNA coding sequence comprises the sequence of:
a) TCG AGT TCC TTG ATG TAG TTC (SEQ ID NO: 2) or a sequence having up to 10 substitutions; or
b) CGA TCG AGT TCC TTG ATG TAG (SEQ ID NO: 3), or a sequence having up to 10 substitutions.
   7. The expression cassette of any one of embodiments 1 to 6, wherein the miRNA coding sequence comprises SEQ ID NO: 4, or a sequence having up to 30 substitutions.
   8. The expression cassette of any one of embodiments 1 to 6, wherein the miRNA coding sequence comprises SEQ ID NO: 5, or a sequence having up to 30 substitutions.
   9. The expression cassette of any one of embodiments 1 to 6, wherein the miRNA coding sequence comprises SEQ ID NO: 11, or a sequence having up to 60 substitutions.
   10. The expression cassette of any one of embodiments 1 to 6, wherein the miRNA coding sequence comprises SEQ ID NO: 12, or a sequence having up to 60 substitutions.
   11. The expression cassette according to any one of embodiments 1 to 10, wherein the regulatory sequences comprise one or more of a promoter, intron, WPRE, and poly A.
   12. The expression cassette according to embodiment 11, wherein the regulatory sequences comprise a CB7 promoter or a Syn promoter.
   13. An adeno-associated virus (AAV) comprising an AAV capsid having packaged therein a vector genome, the vector genome comprising the expression cassette of any of embodiments 1 to 12, flanked by a 5′ AAV ITR and 3′ AAV ITR.
   14. The AAV according to embodiment 13, wherein the AAV capsid is selected from AAV9, AAVhu68, AAV1, and AAVrh91.
   15. The AAV according to embodiment 14, wherein the AAV capsid is AAVhu68.
   16. The AAV according to any one of embodiments 13 to 15, wherein the regulatory sequences comprise a neuronal specific promoter.
   17. The AAV according to embodiment 16, wherein the promoter is a human synapsin promoter.
   18. The AAV according to any one of embodiments 13 to 17, wherein the regulatory sequences comprise a constitutive promoter.
   19. The AAV according to embodiment 18, wherein the promoter is a CB7 promoter.
   20. The AAV according to any one of embodiments 13 to 19, wherein the regulatory sequences comprise a WPRE.
   21. The AAV according to any one of embodiments 13 to 20, wherein the regulatory sequences comprise an intron.
   22. The AAV according to any one of embodiments 13 to 21, wherein the regulatory sequences comprise a rabbit beta globin poly A.
   23. A composition comprising a nucleic acid sequence encoding at least one hairpin forming miRNA that comprises a targeting sequence which binds a target site on the mRNA of human androgen receptor, operably linked to regulatory sequences which direct expression of the nucleic acid sequence in the subject, wherein the miRNA inhibits expression of human androgen receptor.
   24. The composition according to embodiment 23, wherein the miRNA targets the following site on human androgen receptor mRNA: GAA CTA CAT CAA GGA ACT CGA (SEQ ID NO: 1).
   25. A pharmaceutical composition comprising the expression cassette according to any one of embodiments 1 to 12, an AAV according to embodiment 13 to 22, or a composition according to embodiment 23 or 24, and a pharmaceutically acceptable aqueous suspending liquid, excipient, and/or diluent.
   26. A method for treating a subject having Spinal and Bulbar Muscular Atrophy (SBMA) comprising delivering an effective amount of the expression cassette according to any one of embodiments 1 to 12, an AAV according to embodiment 13 to 22, or a composition according to embodiment 23 or 25 to a subject in need thereof.
   27. Use of an expression cassette according to any one of embodiments 1 to 12, an AAV according to embodiment 13 to 22, or a composition according to embodiment 23 or 25 for treatment of a patient having Spinal and Bulbar Muscular Atrophy (SBMA).
   28. The use according to embodiment 27, wherein the composition is formulated to be administered intrathecally at a dose of 1×10¹⁰ GC/g brain mass to 3.33×10¹¹ GC/g brain mass of the rAAV.
   29. The use according to any one of embodiments 27 or 28, wherein the patient is a human adult and is administered a dose of 1.44×10¹³ to 4.33×10¹⁴ GC of the rAAV.
   30. The use according to any one of embodiments 27 to 29, wherein the rAAV is delivered intrathecally, via intracerebroventricular delivery, or via intraparenchymal delivery.
   31. The use according to any one of embodiments 27 to 29, wherein the composition is administered as a single dose via a computed tomography-(CT-) guided sub-occipital injection into the cisterna magna (intra-cisterna magna).
   32. The use according to any one of embodiments 19 to 25, wherein the patient has SBMA.
   33. A method of treating a human patient with spinal and bulbar muscular atrophy, comprising delivering to the central nervous system (CNS) a recombinant adeno-associated virus (rAAV) having an AAV capsid of adeno-associated virus hu.68 (AAVhu.68), said rAAV further comprising a vector genome packaged in the AAV capsid, said vector genome comprising AAV inverted terminal repeats, a nucleic acid sequence encoding at least one hairpin forming miRNA that comprises a targeting sequence which binds a target site on the mRNA of human androgen receptor, wherein the miRNA inhibits expression of human androgen receptor, and regulatory sequences which direct expression of the miRNA.
   34. The method according to embodiment 33, wherein the patient is administered an expression cassette according to any one of embodiments 1 to 12, an AAV according to embodiment 13 to 22, or a composition according to embodiment 23 or 25.
   35. The method according to any one of embodiments 33 or 34, wherein the patient is administered a dose of 1×10¹⁰ GC/g brain mass to 3.33×10¹¹ GC/g brain mass of the rAAV intrathecally.
   36. The method according to any one of embodiments 33 to 35, wherein the patient is a human adult and is administered a dose of 1.44×10¹³ to 4.33×10¹⁴ GC of the rAAV.
   37. The method according to any one of embodiment 33 to 36, wherein the rAAV comprising the miR coding sequence is delivered intrathecally, via intracerebroventricular delivery, or via intraparenchymal delivery.
   38. The method according to any one of embodiments 33 to 37, wherein the rAAV is administered as a single dose via a computed tomography-(CT-) guided sub-occipital injection into the cisterna magna (intra-cisterna magna).
   39. The method according to any of embodiments 27 to 35, wherein the patient has SBMA.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The following examples are illustrative only and are not intended to limit the present invention.

### Example 1: Screening of Androgen Receptor (AR)-Targeting miRNAs In Vitro

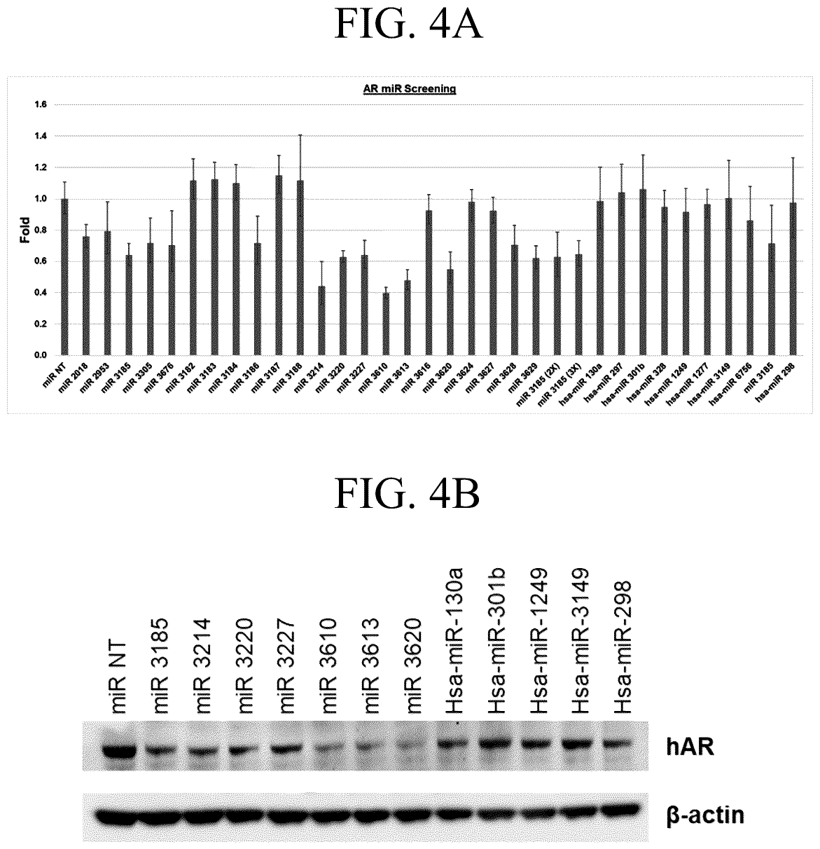

HEK293 cells were transfected with Block-iT plasmids. The Block-IT plasmids contained a CMV promoter, emGFP, cloning site for miRNA and TK polyA and miRNAs were designed using Block-iT online software. The miRNA flanking region was based on miR155. Cell lysates were extracted and prepped for RNA extraction and qPCR or Western blotting. RNA extracted from cells was reverse transcribed into cDNA and qPCR was performed using a TaqMan assay against AR. The graph shows the knockdown levels of AR after transfection with the different miRNAs. The qPCR highlighted miR 3160 as the most efficient miRNA to knockdown AR in vitro (FIG. 4A). Protein analysis on a limited number of miRNA confirmed that miR 3610 effectively knockdown protein expression of AR (FIG. 4B).

### Example 2: Evaluation of Route of Administration

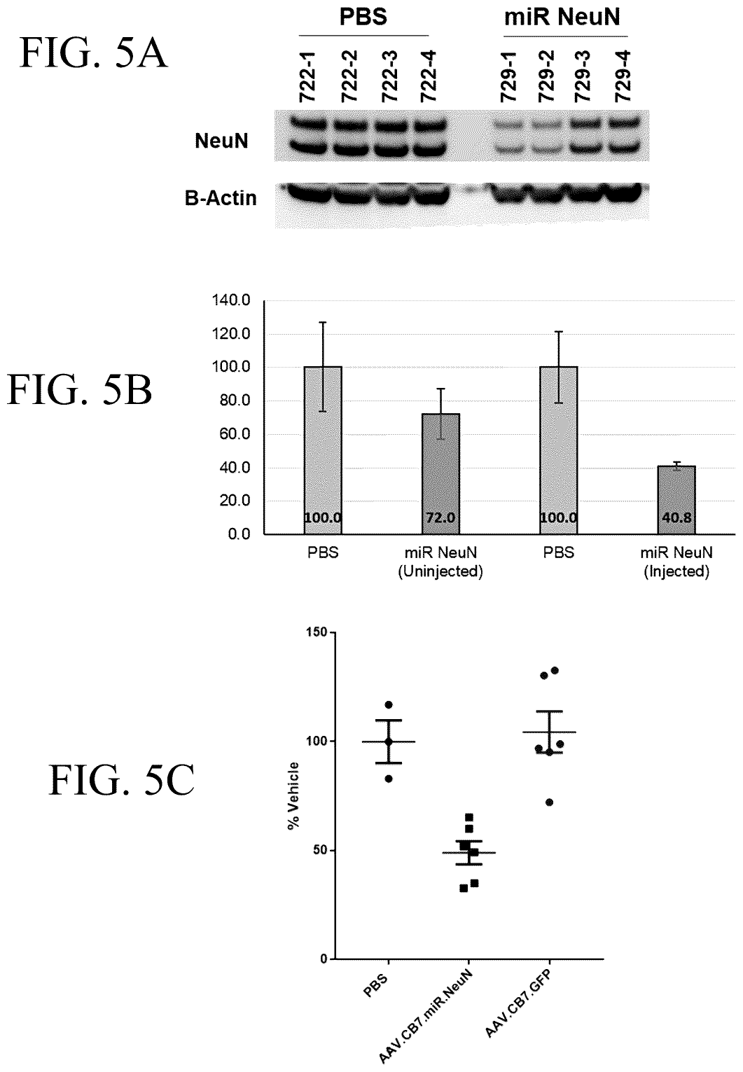

To evaluate route of administration, wildtype adult mice were injected via tail vein and neonatal mice were injected via intracerebroventricular with the following: PBS, AAV.CB7.miR_NeuN (3×10¹¹ GC) or AAV.CB7.GFP (3×10¹¹ GC). Mice were sacrificed 14 days post injection. The brains and spinal cord were harvested, homogenized, and processed for Western blotting. NeuN protein levels were reduced in both the neonatal mice and adult mice that were injected with miR NeuN (FIG. 5A-5C).

### Example 3: Evaluation of Knockdown of the Androgen Receptor In Vivo

Mice were administered AAV9.PHP.eB.CB7.CI.hARmiR3610.WPRE.rBG (3×10¹¹ GC in 100 μL) or PBS via tail vein injection. The mice were sacrificed 14 days post injection. The brains and spinal cords were harvested and processed for RNA and

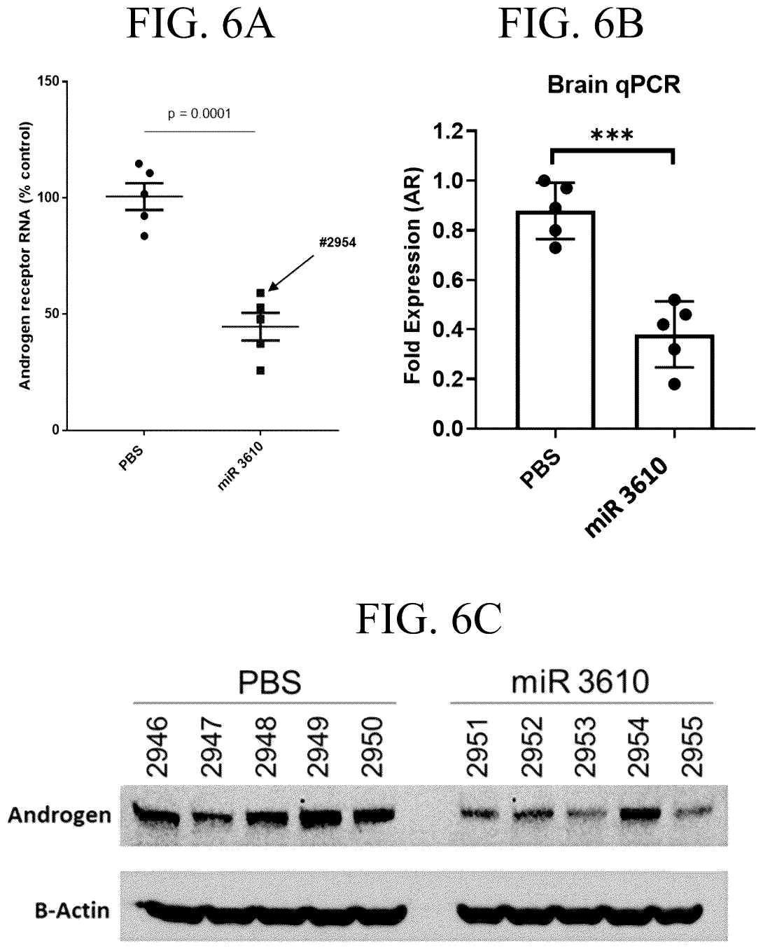

Western blotting. miR 3610 cross reacts with human and mouse AR mRNA. RNA was isolated, cDNA was synthesized, and qPCR was performed using TaqMan primers against hAR. All mice in the miR 3610-injected group showed a 40% reduction in AR mRNA levels compared to the PBS-injected group (FIG. 6A, 6B). AR protein levels were also reduced in the miR 3610-injected group compared to the PBS-injected group (FIG. 6C).

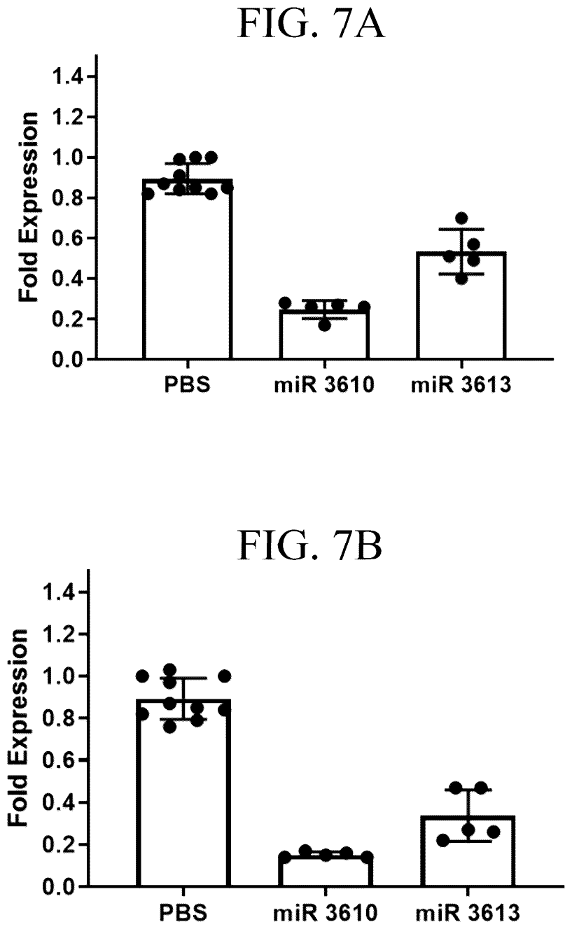

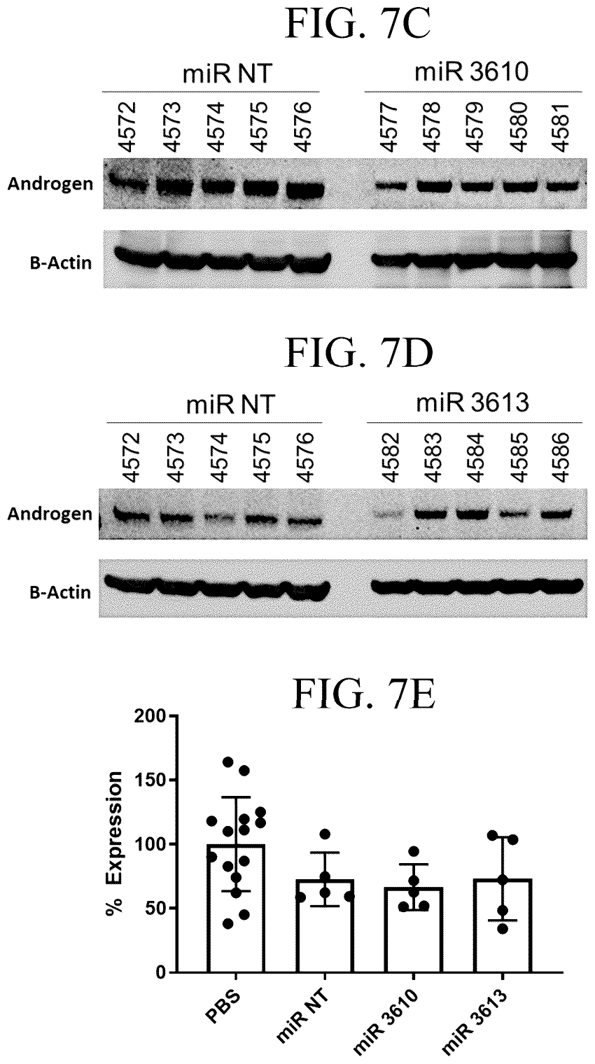

In vitro screening of the different miRNAs also identified miR 3613 as a potential therapeutic target to knockdown AR. To evaluate miR 3613 in vivo as compared with miR 3610, mice were administered the following vectors: AAV9.PHP.eB.CB7.Cl.hARmiRNT.WPRE.rBG, AAV9.PHP.eB.CB7.Cl.hARmiR3610.WPRE.rBG, AAV9.PHP.eB.CB7.Cl.hARmiR3610.WPRE.rBG or PBS via tail vein. Mice were sacrificed at day 14. The brains and spinal cords were harvested and processed for RNA and Western blotting. Both miRNAs elicited a reduction in AR mRNA levels in brain (FIG. 7A) and spinal cord (FIG. 7B). Similar results were seen with both miRNAs for protein levels in the brain (FIG. 7C-E), although miR 3610 had a more pronounced effect on gene and protein levels compared to miR 3613.

### Example 4: Evaluation of Different Promoters

This study evaluated four different AAV9-PHP.eB vectors that were identical except that they included two different promoters (CB7 or hSyn) expressing either a non-targeting artificial miRNA (miR.NT) or an AR-targeted artificial miRNA (miR3610). The CB7 promoter (included in GTP-211) is a ubiquitous chicken β-actin promoter and was evaluated because it results in a high level of expression in any CNS cell type. The hSyn promoter is the human synapsin promoter, which results in a high level of expression specifically in neurons and would be expected to minimize expression in non-neuronal cell types. miR.NT is a non-targeting artificial miRNA that is expected to have few to no sequence similarities with other expressed genes in the mouse, and serves as a negative control vector. The hAR.miR3610 (included in GTP-211) is an artificial miRNA sequence targeting human AR mRNA and was chosen based on data previously collected.

Adult male wild type mice (6-8 weeks old) received a single IV administration of AAV9.PHP.eB.CB7.CI.miR.NT.WPRE.rBG, AAV9.PHP.eB.CB7.CI.hARmiR3610.WPRE.rBG, AAV9.PHP.eB.Syn.PI.miR.NT.WPRE.bGH, or AAV9.PHP.eB.hSyn.PI.hARmiR3610.WPRE.bGH at a dose of 3.0×10¹¹ GC. On Day 14, mice were necropsied. Spinal cord was collected to evaluate mouse AR mRNA expression (TaqMan qPCR).

AAV administration was well-tolerated, and all mice survived to the scheduled necropsy.

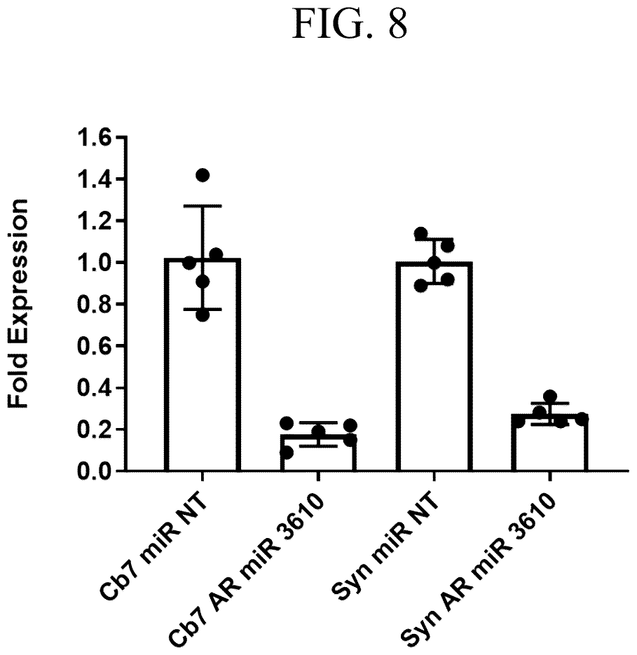

As shown in FIG. 8, administration of AAV9.PHP.eB vectors expressing the non-targeting artificial miRNA (miR.NT) did not impact AR mRNA expression, and similar levels of expression were observed with both the CB7 and hSyn promoters. In comparison, mice treated with AAV9.PHP.eB vectors expressing the artificial miRNA sequence (hAR.miR3610) targeting human AR mRNA demonstrated knockdown of the mouse AR mRNA transcript, indicating that both the CB7 and hSyn promoters resulted in robust expression of hAR.miR3610.

### Example 5: In Vivo SBMA AR97Q Transgenic Mice Studies

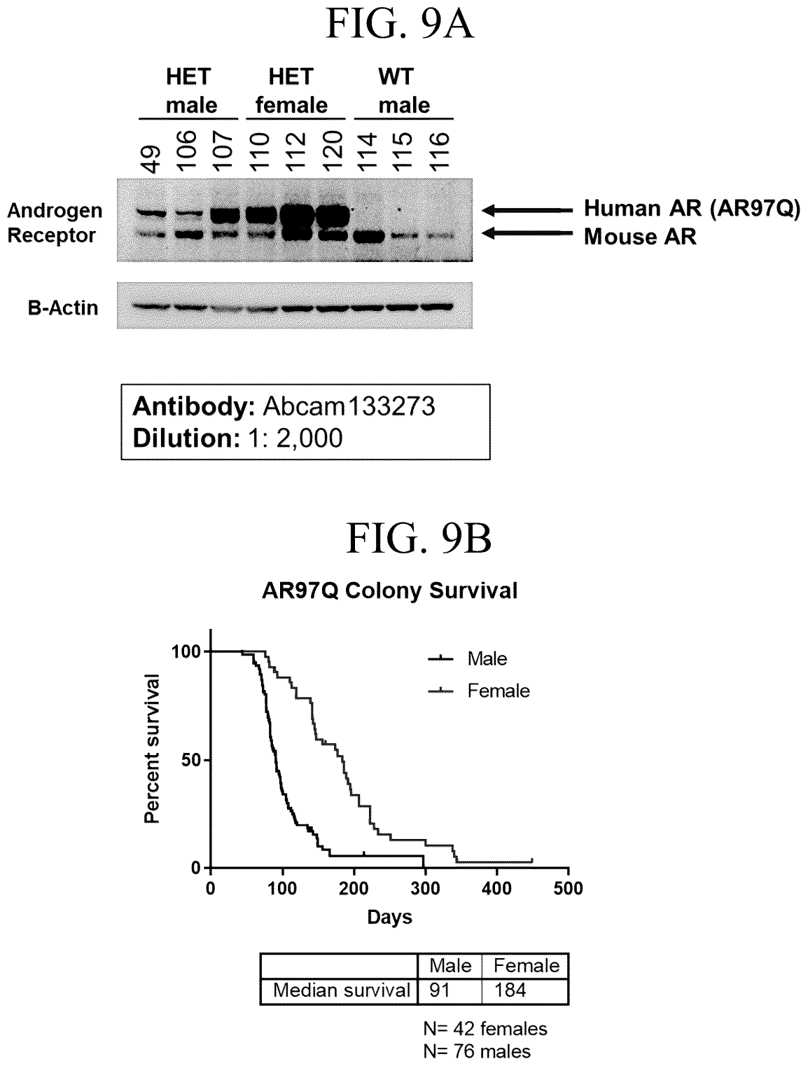

SBMA transgenic mice have been described by Katsuno et al (Neuron. 2002 Aug. 29;35(5):843-54. doi: 10.1016/s0896-6273(02)00834-6, incorporated herein by reference). The mouse model carries a full-length AR containing 97 CAGs. To assess the level of AR expressed in AR97Q transgenic mice, spinal cords were harvested from wildtype male mice and heterozygous male and female mice. The spinal cords were processed for Western blotting. Male and female heterozygous mice displayed robust levels of hAR(AR97Q), whereas the WT male mice displayed no expression of hAR. All mice displayed varying levels in mAR (FIG. 9A). Survival was also tracked in this colony. The plot indicated a sharp drop in survival for males with a median survival of 92 days, whereas a gradual decline in survival was observed for the females with a median survival of 192 days (FIG. 9B).

To evaluate the effects of miR 3610 in the transgenic line, Adult male SBMA mice (5-6 weeks old) received a single IV administration of AAV9.PHP.eB.CB7.CI.hARmiR3610.WPRE.rBG at a dose of 3.0×10¹¹ GC via the tail vein. Additional age-matched male SBMA mice remained uninjected as controls. Animals were checked daily for viability (survival). At the humane endpoint, mice were necropsied, and brains were collected to evaluate the expression of mutant human AR protein and endogenous mouse AR protein by Western blot.

AAV administration was well-tolerated. All mice reached a humane endpoint due to disease progression characterized by a body condition score of ⅖ or less, inability of the mouse to right itself, or paralysis of two or more limbs.

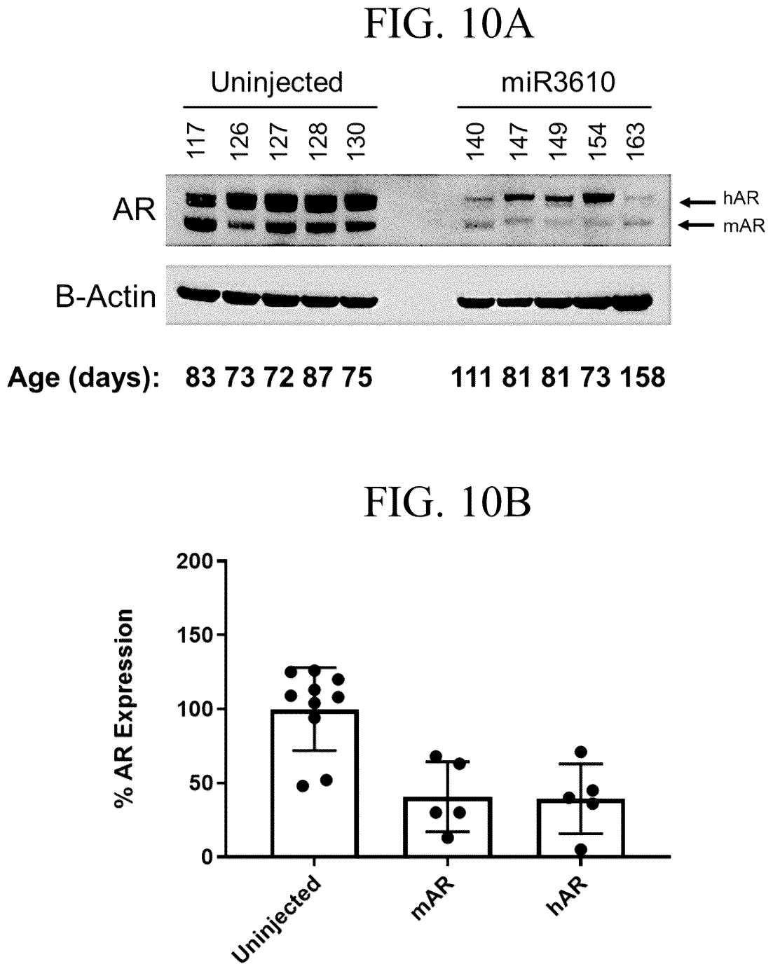

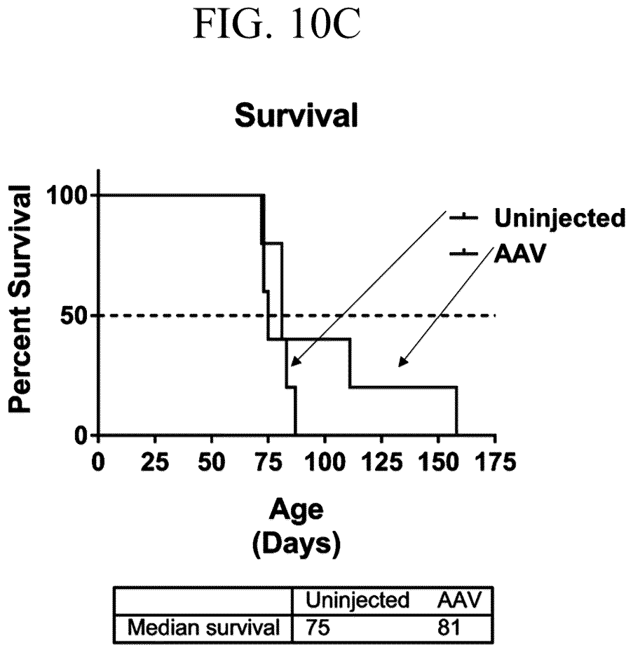

The median survival of AAV-treated SBMA mice was 81 days of age, whereas the median survival of uninjected control SBMA mice was 75 days of age (FIG. 10C). The difference in survival between the AAV-treated SBMA mice and uninjected controls was not statistically significant.

In the brain, substantial knockdown of both endogenous mouse AR protein and mutant human AR protein was observed by Western blot in AAV-treated SBMA mice, but not uninjected controls (FIG. 10A). Western blot quantification revealed that AAV-treated SBMA mice exhibited an approximately 2-fold reduction in expression of endogenous mouse AR protein and mutant human AR protein in the brain compared to uninjected SBMA control mice (FIG. 10B).

Among the AAV-treated SBMA mice, longer survivals were observed in animals that exhibited greater knockdown of the mutant human AR protein. Of note, Animal 140 and Animal 163 exhibited the greatest reduction in mutant human AR protein expression and had survivals of 111 and 158 days, respectively (FIG. 10A). In contrast, Animals 147, 149, and 154 demonstrated higher expression of the mutant human AR protein and had shorter survivals ranging from 73 to 81 days (FIG. 10A,10C).

Juvenile male SBMA mice (3 weeks of age) received a single IV administration of AAV9.PHP.eB.CB7.CI.hARmiR3610.WPRE.rBG at a dose of 3.0×10¹¹ GC via the retro-orbital vein. Natural history data from the SBMA mouse colony or uninjected SBMA mice served as historical controls. Animals were checked daily for viability (survival). At the humane endpoint, mice were necropsied, and brains were collected to evaluate the expression of mutant human AR protein and endogenous mouse AR protein by Western blot.

AAV administration was well-tolerated. All mice reached a humane endpoint due to disease progression characterized by a body condition score of ⅖ or less, inability of the mouse to right itself, or paralysis of two or more limbs.

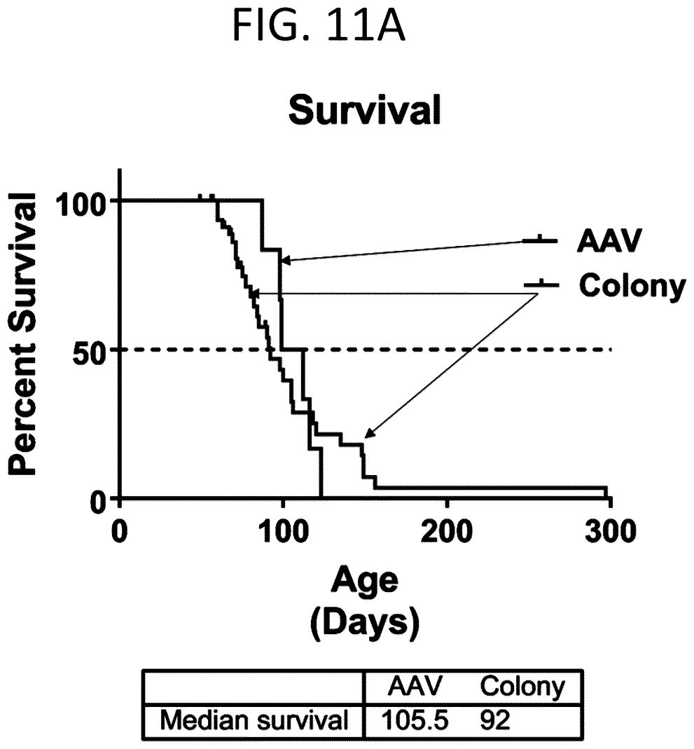

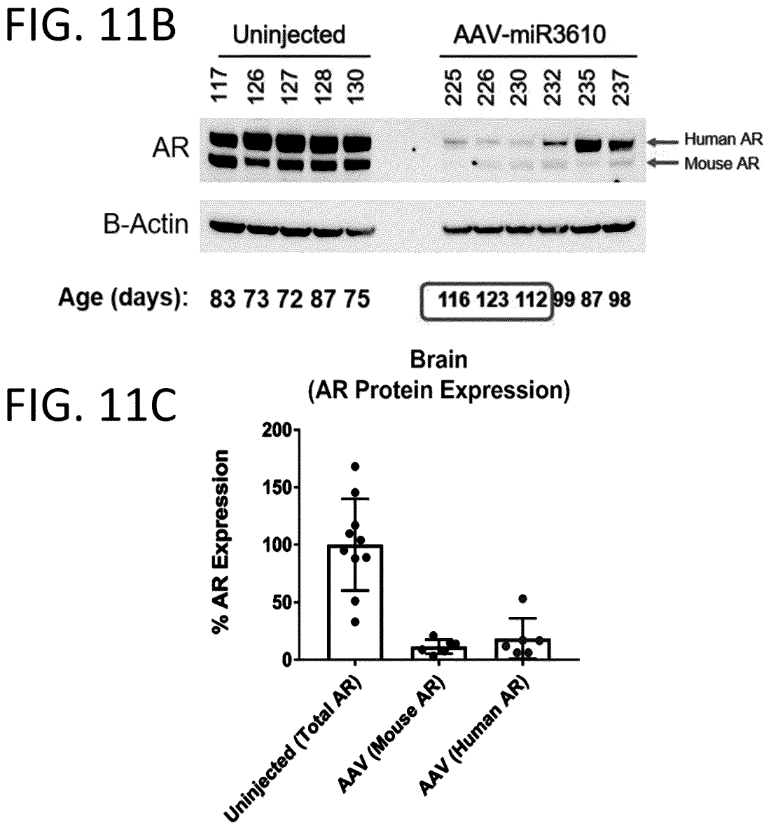

The median survival of AAV-treated SBMA mice was 105.5 days, whereas uninjected historical control SBMA mice had a median survival of 92 days (FIG. 11A). The difference in survival between the AAV-treated SBMA mice and uninjected historical controls was not statistically significant.

In the brain, substantial knockdown of both endogenous mouse AR protein and mutant human AR protein was observed by Western blot in AAV-treated SBMA mice, but not uninjected historical controls (FIG. 11B). Western blot quantification revealed that AAV-treated SBMA mice exhibited an approximately 5-fold and 8-fold reduction in expression of endogenous mouse AR protein and human mutant AR protein, respectively, compared to uninjected SBMA historical control mice (FIG. 11C).

Among the AAV-treated SBMA mice, longer survivals were observed in animals that exhibited greater knockdown of mutant human AR protein. Of note, Animals 225, 226, and 230 exhibited the greatest reduction in mutant human AR protein expression and had survivals ranging from 112 to 123 days (FIG. 11B). In contrast, Animals 232, 235, and 237 exhibited a less substantial reduction in mutant human AR protein expression and had shorter survivals ranging from 87 to 99 days (FIG. 11B).

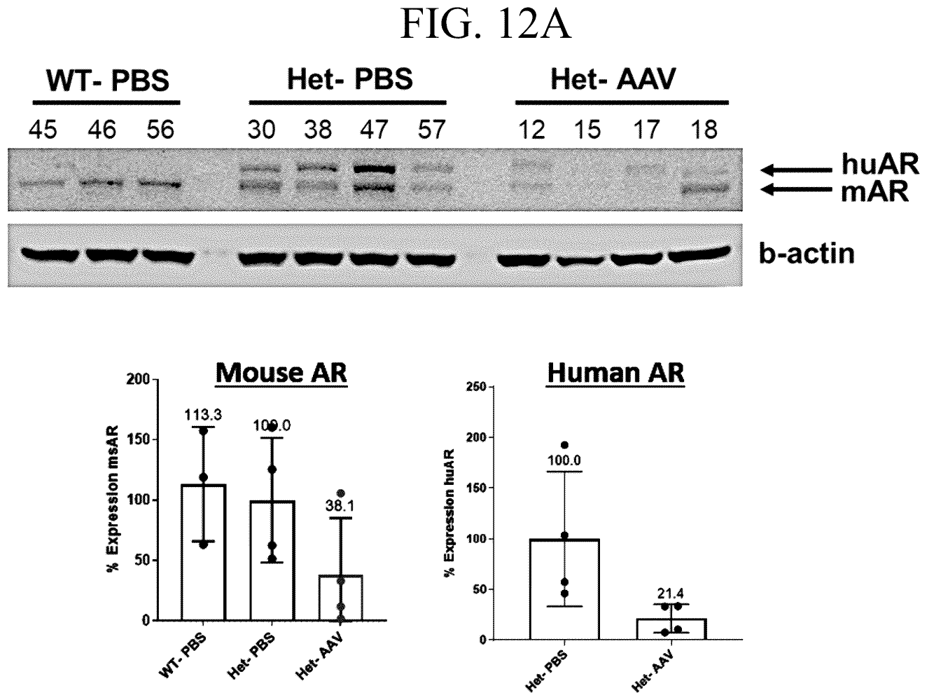

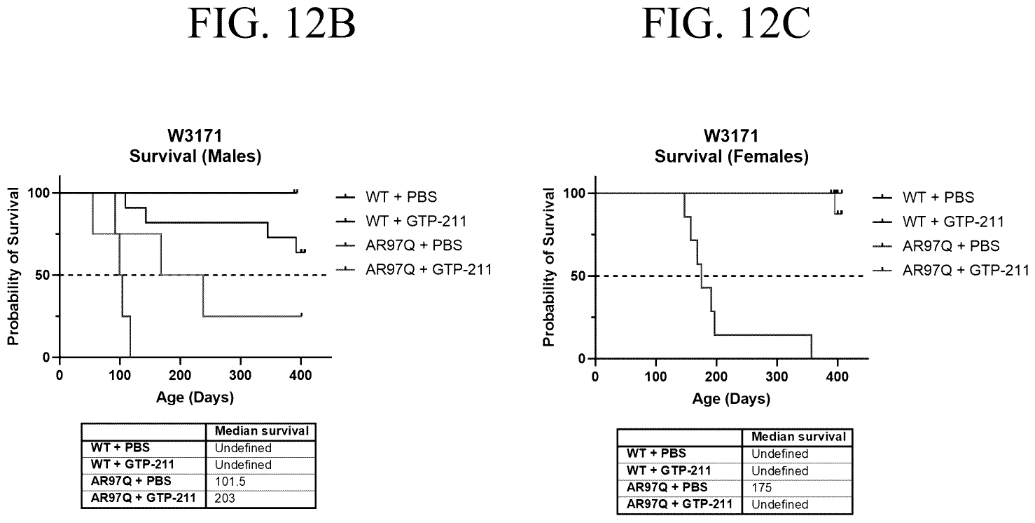

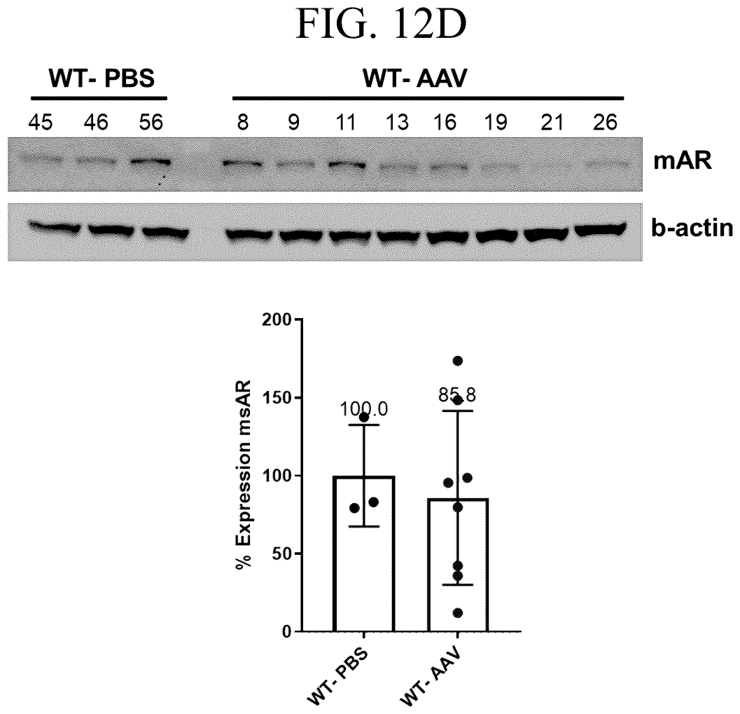

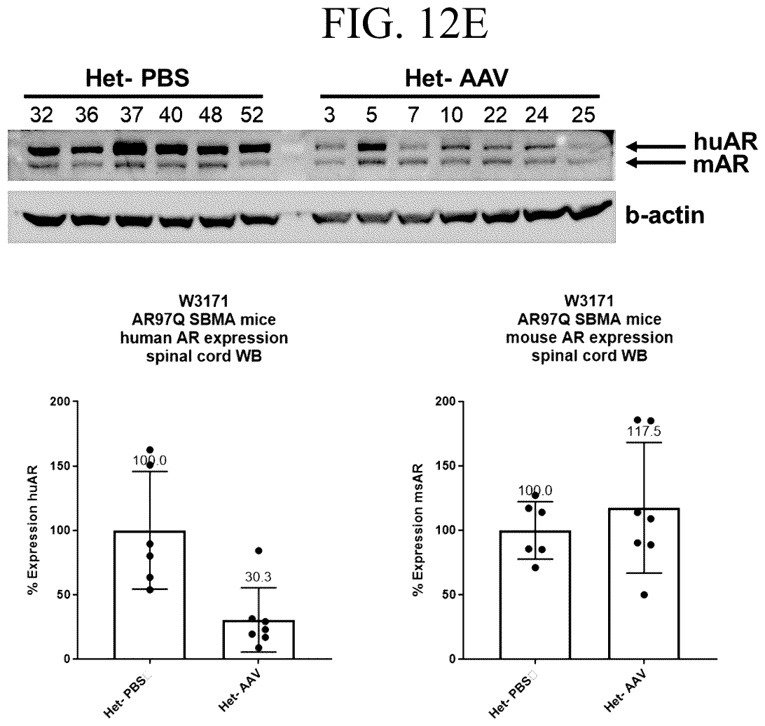

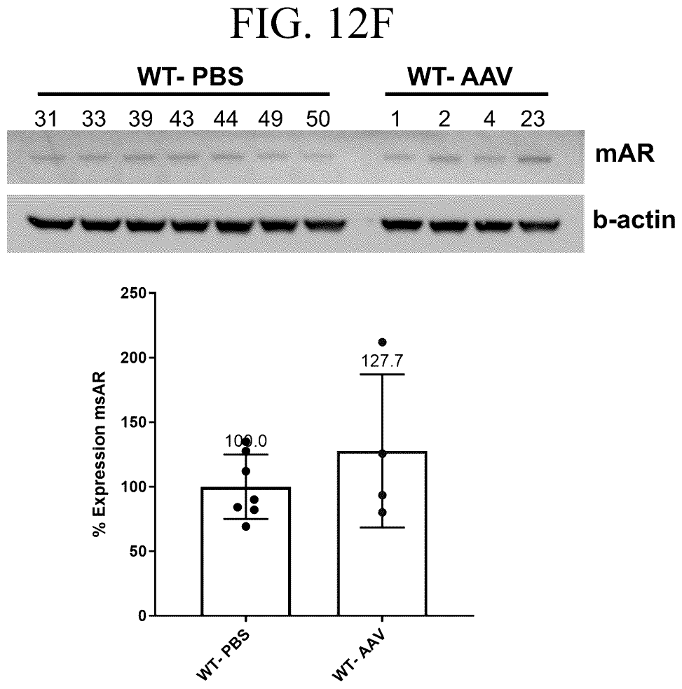

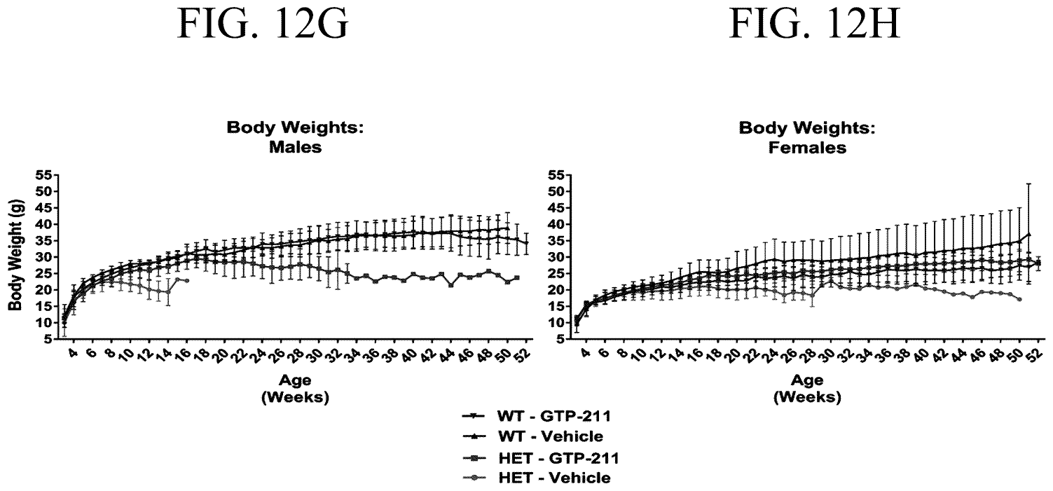

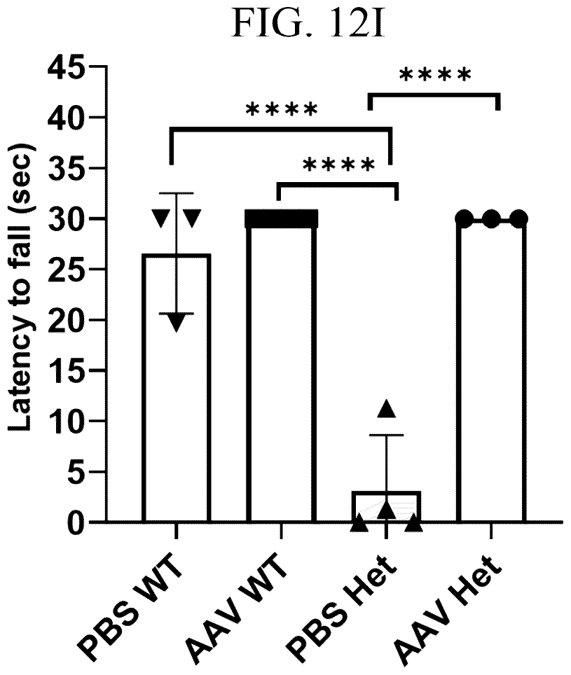

Neonatal (PND 0-1) male and female SBMA mice received a single IV administration of either AAVhu68.CB7.CI.hARmiR3610.WPRE.rBG at a dose of 3.0×10¹¹ GC or vehicle (PBS) via the temporal vein. Additional age-matched male and female C57BL/6J (wild type) received a single IV administration of either AAVhu68.CB7.CI.hARmiR3610.WPRE.rBG at a dose of 3.0×10¹¹ GC or vehicle (PBS) via the temporal vein as controls and because genotypes could not be confirmed until after weaning around PND 21. Animals are checked daily for viability (survival), and body weights are measured weekly. Male mice from both treatment groups underwent the wire hang test at approximately 90 days of age. At the humane endpoint, mice are necropsied, and brains are collected to evaluate mutant human AR protein and endogenous mouse AR protein expression by Western blot. Expression of AR protein is shown in FIG. 12A.

AAV administration was well-tolerated based on daily viability checks. All SBMA mice that have been necropsied to date reached a humane endpoint due to disease progression (defined as a body condition score of ⅖ or less, inability of the mouse to right itself, or paralysis of two or more limbs) except for ¾ AAV-treated male SBMA mice that were found dead on Days 55, 168, and 238 due to an undetermined cause. All wild type mice are still alive, except for 2/11 AAVhu68.CB7.CI.hARmiR3610.WPRE.rBG-treated male wild type mice that were found dead on Days 109 and 143 due to an undetermined cause.

AAVhu68.CB7.CI.hARmiR3610.WPRE.rBG administration resulted in a substantial increase in median survival of both male and female SBMA mice when compared to sex-match vehicle-treated SBMA control mice. Among male mice, the median survival of vehicle-treated SBMA mice was 101.5 days, while a significantly longer median survival of 203 days was observed for GTP-211-treated SBMA mice. Among female mice, the median survival of vehicle-treated SBMA mice was 175 days, while all AAVhu68.CB7.CI.hARmiR3610.WPRE.rBG-treated SBMA mice (N=8/8) are currently alive at ages currently ranging from 366-373 days old, demonstrating a significant increase in survival following AAVhu68.CB7.CI.hARmiR3610.WPRE.rBG treatment (FIGS. 12B and 12C).

Both male and female GTP-211-treated SBMA mice exhibited improved body weight gain and maintenance over time compared to sex-matched vehicle-treated SBMA controls, indicating an improvement in the body wasting phenotype associated with disease progression (FIG. 12G, 12H).

At approximately 90 days of age, the wire hang test was performed on male mice to assess muscle strength and coordination. Vehicle-treated SBMA mice exhibited significantly reduced fall latencies compared to vehicle- and AAVhu68.CB7.CI.hARmiR3610.WPRE.rBG-treated wild type controls. In contrast, AAVhu68.CB7.CI.hARmiR3610.WPRE.rBG administration led to a significant increase in fall latencies in SMBA mice compared to the vehicle-treated SBMA mice. Moreover, AAVhu68.CB7.CI.hARmiR3610.WPRE.rBG-treated animals performed this behavioral assay as well as wild type mice, indicating that AAVhu68.CB7.CI.hARmiR3610.WPRE.rBG fully preserved muscle strength and coordination in SBMA mice (FIG. 12I).

### Example 6: Evaluation of MIR3610 in NHP

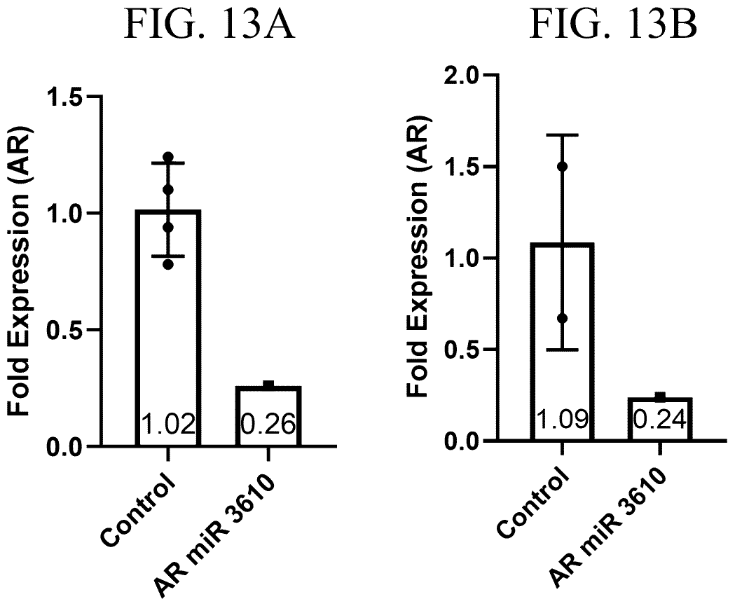

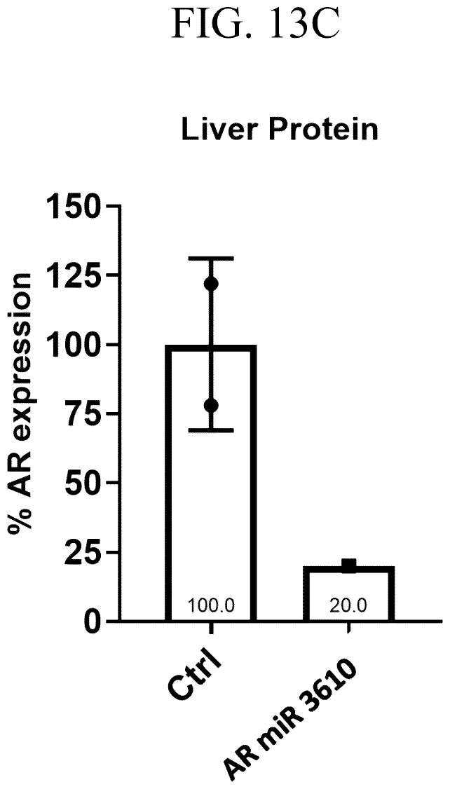

5-year old male rhesus macaque was administered with 3×10¹³ GC of AAVhu.68.CB7.CI.hARmiR3610.WPRE.rBG via intra cisterna magna (ICM). Control samples were derived from uninjected NHP samples. The NHP was sacrificed at day 35. The spinal cord was harvested, fixed with formalin, and embedded for laser capture microdissection (LCM). The formalin-fixed paraffin embedded blocks were cut and placed on PEN membrane slides suitable for LCM. The motor neurons were cut from the spinal cord sections. RNA was extracted and qPCR was performed. The liver was also harvested and processed for RNA isolation and qPCR. Motor neurons (FIG. 13A) and liver (FIG. 13B) both displayed a significant reduction (approximately 75%) in AR mRNA levels after injection with miR 3610. AR protein expression was also reduced after injection with miR 3610 (FIG. 13C). In-life safety endpoints including cage side observations, serum chemistry, complete blood counts, nerve conduction studies and CSF chemistry and cytology demonstrated no evidence of vector-related toxicity after 35 days. No significant pathological findings were observed in any tissues, including DRGs.

### Example 7: Mouse MED Study

This planned GLP-compliant pharmacology study aims to evaluate the efficacy and determine the MED of IV-administered AAVhu.68.CB7.CI.hARmiR3610.WPRE.rBG in the male SBMA mouse model (AR-97Q mice).

This study will evaluate N=60 neonatal (PND 0-1) male SBMA mice and N=12 age-matched male wild type C57BL/6J mice as controls. The study will include one necropsy time point (180 days). Four dose levels of AAVhu.68.CB7.CI.hARmiR3610.WPRE.rBG will be evaluated using IV administration. The dose levels will be selected based on POC efficacy data in the ongoing study evaluating treatment of neonatal SBMA mouse, in addition to the completed pilot safety and pharmacology study conducted in adult rhesus macaque NHPs. The dose levels evaluated will bracket the anticipated clinical doses.

While IV administration is currently planned for this study, ongoing pilot studies in neonatal mice are evaluating the ICV route for vector delivery directly into the CSF to target the disease-relevant cell type (spinal motor neurons) (data not yet available). If similar spinal motor neuron targeting can be achieved with ICV administration as systemic administration, this intrathecal route will be employed for this study to more closely model the intended clinical ROA (ICM administration).

### Example 8: GLP NHP Pharmacology/Toxicity Study

A 180 day GLP-compliant toxicology study will assess the safety, tolerability, pharmacology (artificial miRNA-mediated knockdown of macaque AR), biodistribution, and excretion profile of following a single ICM administration at a low dose, mid-dose, or high dose (N=4/dose) to adult male rhesus macaques (5-7 years). Additional age-matched male NHPs will be administered vehicle (ITFFB) as a control (N=2).

14 adult male rhesus macaques receive one of 3 doses (4 NHP per dose; 2 NHP receiving vehicle) of AAVhu.68.CB7.CI.hARmiR3610.WPRE.rBG via image guided intra cisterna magna (ICM). Half are sacrificed at 90 days and the others are sacrificed at 180 days. The spinal cord is harvested, fixed with formalin, and embedded for laser capture microdissection (LCM). The formalin-fixed paraffin embedded blocks are cut and placed on PEN membrane slides suitable for LCM. The motor neurons are cut from the spinal cord sections. RNA is extracted and qPCR is performed. The liver is also harvested and processed for RNA isolation and qPCR. The following tests are also performed: CBC/chem/Coags; CSF chemistry and cytology; Blinded neurological exams; Nerve conduction velocity testing; and Histopathology.

### Example 9: Evaluation of ICV Delivery of AAVhu.68.CB7.CI.hARmiR3610.WPRE.rBG in SBMA Neonatal Mice

Neonatal SBMA transgenic mice were administered 3e11 GC of AAVhu68.CB7.CI.ARmiR3610.WPRE.rBG via ICV or PBS. Mice were tracked for survival and body weight, and sex and genotypes were determined. Mice were subject to the wirehang test. The brains were harvested at the time of death and processed for Western blotting.

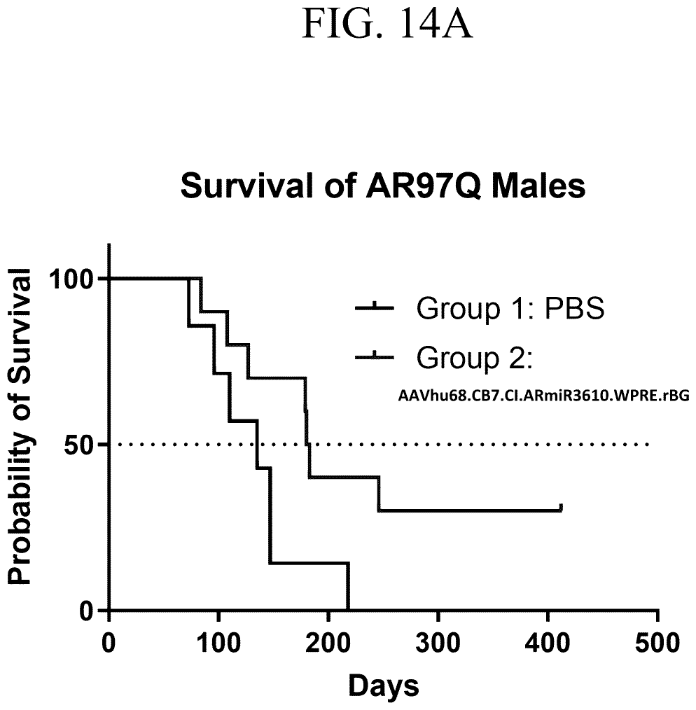

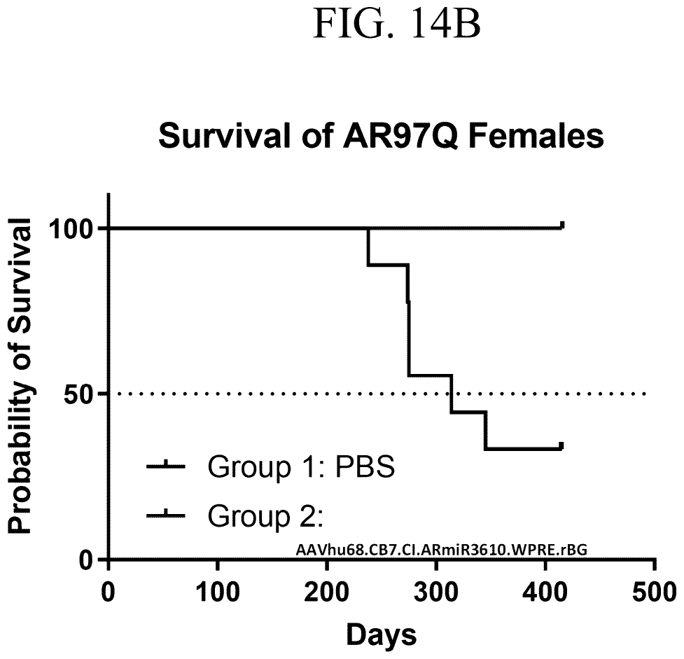

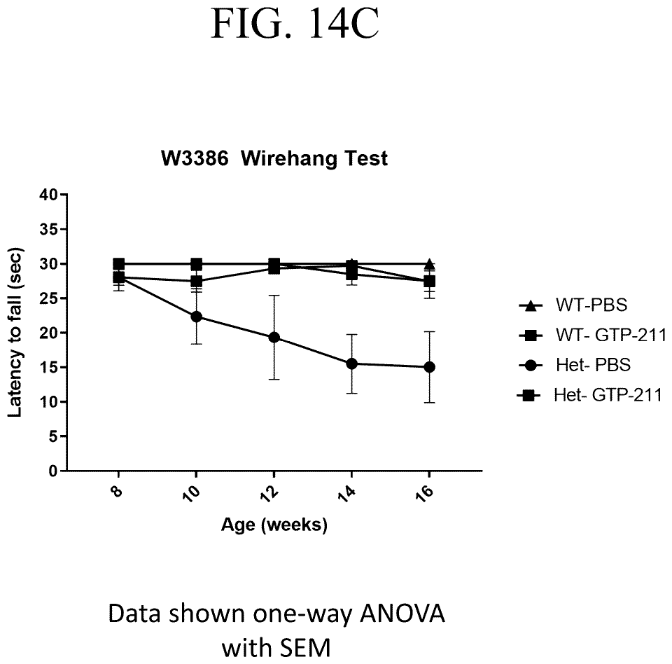

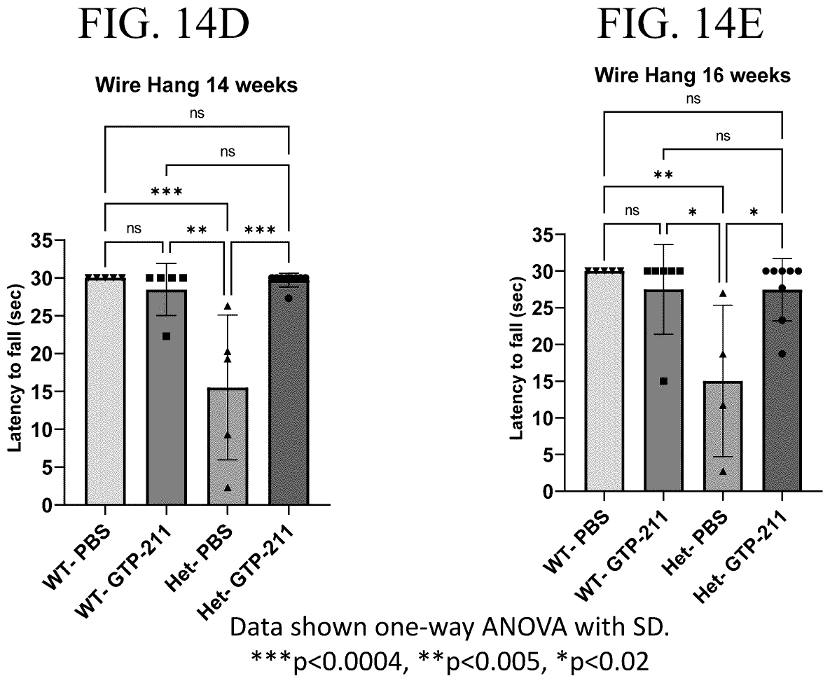

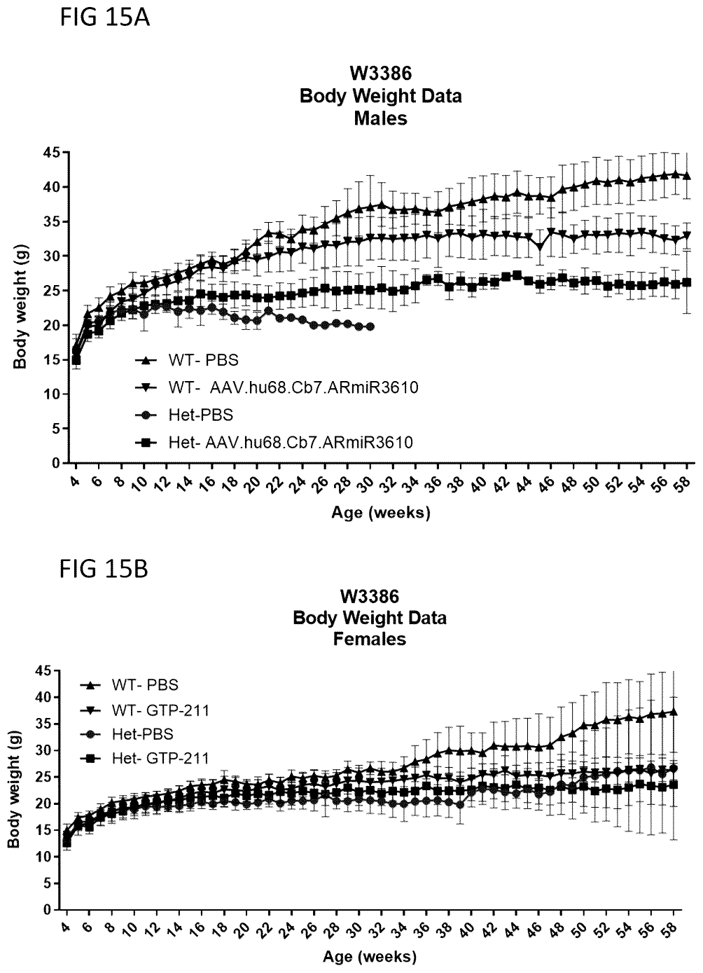

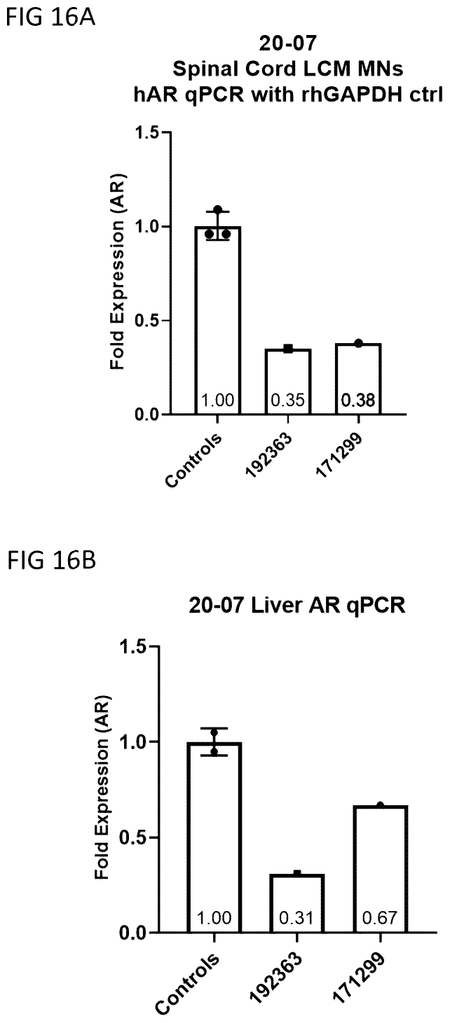

Male SBMA mice had an average lifespan of 135 days for PBS treated mice, an average age of onset of about 150 days, and an average life span of 181.5 days for AAV treated mice (FIG. 14A). Female SBMA mice had an average life span of 314 days for AAV treated mice (FIG. 14B). Body weight for the males and females is shown in FIGS. 15A and 15B, respectively. The average age of onset for PBS treated mice was 80 days for PBS treated and 150 days for AAV treated mice (FIG. 14C). Hets treated with PBS had significantly reduced hang time as compared to PBS-treated WT and AAV-treated Hets (FIG. 14C). Hang time at 14 weeks (FIG. 14D) and 16 weeks (FIG. 14E) was markedly decreased for Hets treated with PBS.

### Example 10: In Vivo NHP Study

| | |
|---|---|
| Species: | Rhesus macaques |
| Age at Injection: | 15-7 yrs old |
| No. and Sex: | 2 males |
| Vector: | GTP-211 |
| Dose: | 3e13 GC |
| Capsid: | AAV9.hu68 |
| Volume: | 1 mL |
| ROA: | Intra-Cisterna Magna (ICM) |

FIGS. 16A and 16B show results of laser-capture microdissection (LCM) of lumbar spinal cord motor neurons (MNs) following in vivo non-human primate injection of AAVhu68.CB7.CI.AR.miR3610.WPRE.rBG. Isolated MNs were RNA extracted and qPCR was performed with AR and rhGAPDH control primers. (FIG. 16A) qPCR was also performed on liver samples, however one of the animals (171299) had pre-existing NAbs that partially blocked expression in liver. FIG. 16B. For each of these NHPs, o cage side observations, no evidence of vector-related toxicity after 35 days. For NHP 1, there were no significant pathological findings in any tissues including DRGs. For NHP2, 1 single DRG with Grade 1 axonal degeneration was observed.

Sp cord with grade 1 degeneration, axon, dorsal white matter tracts, all segments

### Example 11: MED Study

GTP-211 refers to AAVhu.68.CB7.CI.hARmiR3610.WPRE.rBG in the following table, which provides a summary of the efficacy study to determine the minimum effective dose.

| Info | Group 1 | Group 2 | Group 3 | Group 4 | Group 5 | Group 6 |
|---|---|---|---|---|---|---|
| # of mice | 12 | 12 | 12 | 12 | 12 | 12 |
| Sex | M | M | M | M | M | M |
| Age | PND0-1 | PND0-1 | PND0-1 | PND0-1 | PND0-1 | PND0-1 |
| Genotype | HET | HET | HET | HET | HET | WT |
| Treatment | GTP-211 | GTP-211 | GTP-211 | GTP-211 | Vehicle | |
| ROA | Unilateral ICV | | | | | |
| Dose (GC) | 3.00e9 | 1.00e10 | 3.00e10 | 1.00e11 | NA | NA |
| Volume | 2.5 uL | 2.5 uL | 2.5 uL | 2.5 uL | 2.5 uL | 2.5 uL |

| | |
|---|---|
| Necropsy | Day 180 (6 months) |

| | Group 1 | Group 2 | Group 3 | Group 4 | Group 5 | Group 6 | |
|---|---|---|---|---|---|---|---|
| Info | [symbol] | [symbol] | [symbol] | [symbol] | [symbol] | [symbol] | Totals: |
| Enrollment | 17 | 18 | 12 | 12 | 22 | 12 | 93 |

[symbol]

indicates data missing or illegible when filed

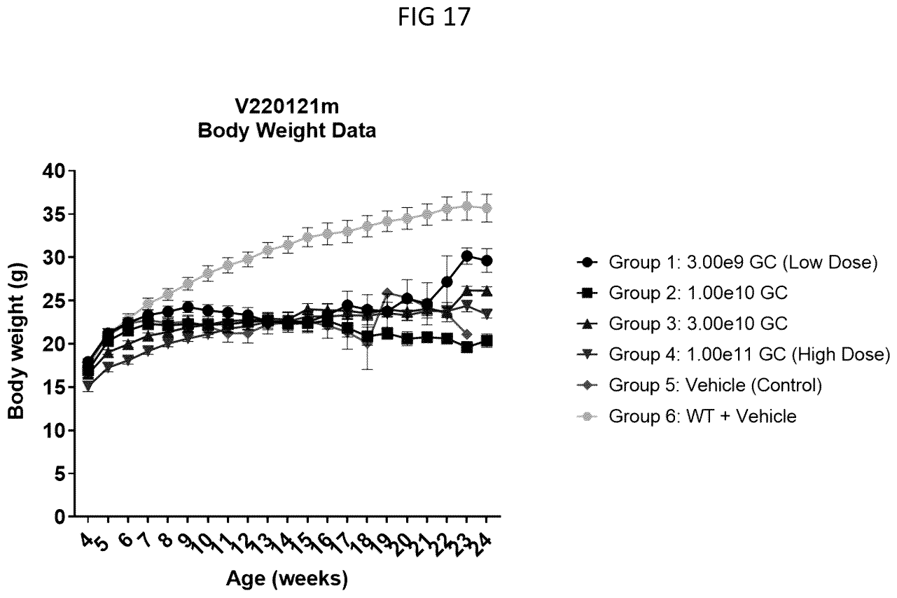

FIG. 17-19C provides interim results from a study (V220121m) designed to study the efficacy of the test vector AAVhu68.CB7.CI.AR.miR3610.WPRE.rBG following intracerebroventricular (ICV) administration in transgenic mice (SBMA (AR97Q) mice to determine the minimum effective dose, over the course of 180 days. The low dose group (Group 1; received 3e9 GC (e=10^{-x})), Group 2 received 1e10 GC; Group 3 received 3e10, and a high dose group (Group 4) received 1e11 GC). Controls included vehicle (no vector; Group 5) and wild-type mice with vehicle (no vector; Group 6)

FIG. 17 provides body weight over the course of 24 weeks.

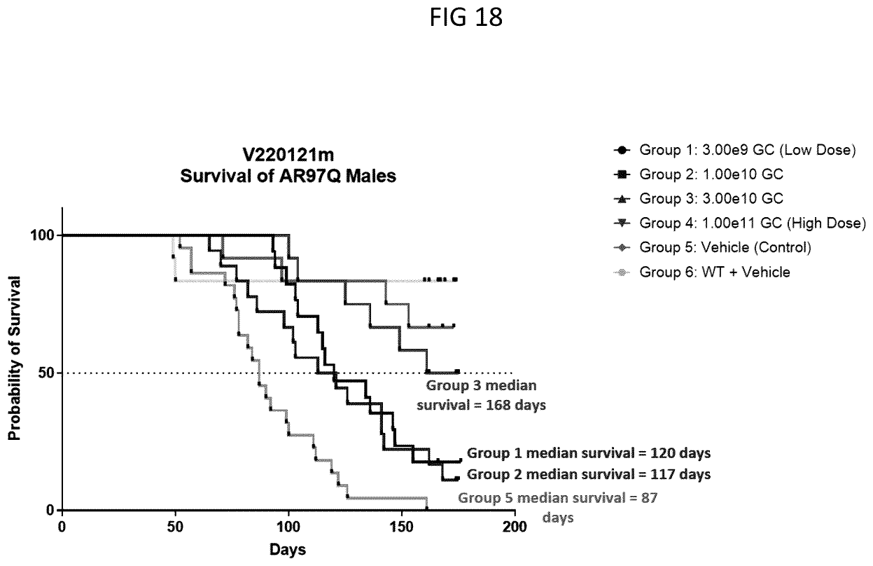

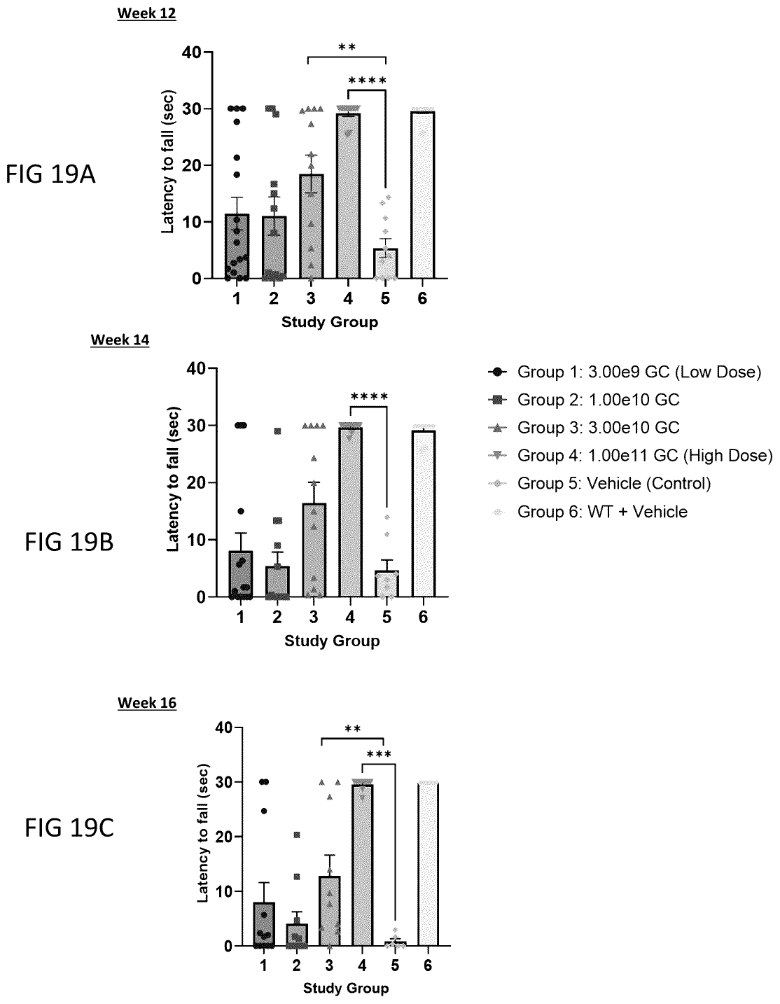

FIG. 18 provides a survival curve for the male transgenic mice. Group 1 shows a median survival of 120 days. Group 2 showed median survival of 117 days. Group 3 showed median survival of 168 days. Group 4 showed survival throughout the test period. FIGS. 19A-19C show the results of the wire hang study for the animals on weeks 12, 14 and 16.

Example 12: Phase I/II Clinical Trial

A Phase I/II clinical trial in humans is proposed. The protocol incorporates recent FDA preIND feedback and guidance for industry for similar applications. The dose escalation/safety study is also designed to allow assessment of key biomarker (thigh muscle volume measured by MRI). Concurrent randomized control (per FDA) provides comparator data.

We will evaluate the safety and tolerability of 2 vector doses. A total of 12 subjects will be enrolled, randomized 2:1 vector:placebo. 8 subjects randomized to vector arm: 2 at low dose, 2 at high dose, 4 at MTD. For first 4 subjects, 30 day data reviewed by DSMB before dosing of subsequent subject. 4 subjects randomized to placebo arm. Analysis of safety and MRI changes at 1 year, with 5-year long-term follow up.

A Danish natural history study of 29 SBMA patients followed for 18 months (Dahlqvist J R, et al. Disease progression and outcome measures in spinobulbar muscular atrophy. Ann Neurol. 2018 November;84(5):754-765 (Incorporated herein by reference)). Dahlqvist showed composite Dixon MRI score of multiple muscles showed highly significant decline, validating use of MRI score in clinical trials. Also demonstrated statistically significant decline in 6MWT, stair climb, and grip strength, although these outcome measures would require greater power in interventional trials.

### Example 13: NHP toxicity and Safety Study

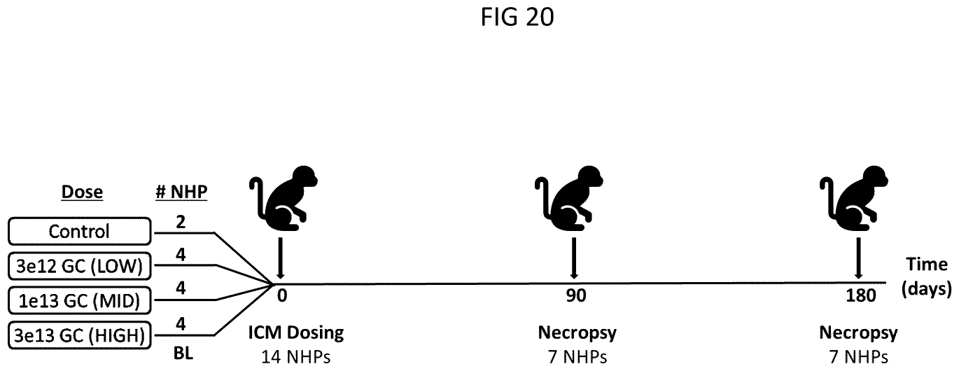

14 rhesus macaques received one of three doses of AAVhu68.CB7.CI.AR.miR3610.WPRE.rBG (3e12 (low), 1e13 (mid), or 3e13 (high)), or served as controls (FIG. 20). No complications were seen from injections. One NHP at high dose presented with abnormal NCV, which was preferentially displayed by one wrist.

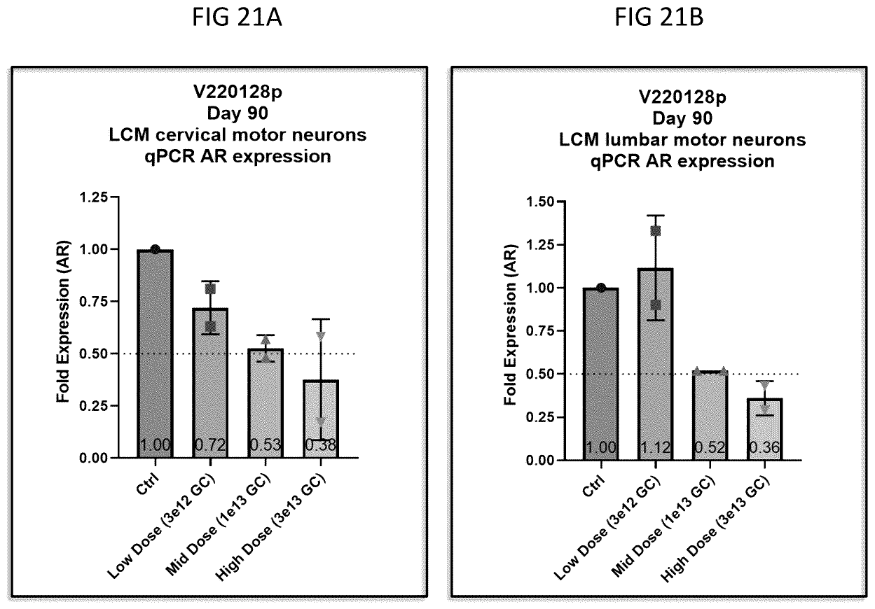

Laser capture microdissection of cervical motor neurons (FIG. 21A) and lumbar motor neurons (FIG. 21B) was utilized for RNA analysis. Both tissues showed dose response knockdown of androgen receptor. No overt toxicity was observed in nonhuman primates, with mild pathology in dorsal root ganglia.

All documents cited in this specification are incorporated herein by reference, as is the Sequence Listing labeled “23-10249.US Seq-Listing”. In addition, International Patent Application No. PCT/US22/24415, filed Apr. 12, 2022, and U.S. Provisional Application Nos. 63/293,505, 63/187,883, and 63/173,885 are incorporated herein by reference. Further, U.S. Provisional Application Nos. 63/381,938 and 63/415,610 are incorporated herein by reference. While the invention has been described with reference to particular embodiments, it will be appreciated that modifications can be made without departing from the spirit of the invention. Such modifications are intended to fall within the scope of the appended claims.

1. An adeno-associated virus (AAV) comprising an AAVhu68 capsid having packaged therein a vector genome, the vector genome comprising an expression cassette comprising a nucleic acid sequence encoding at least one hairpin forming miRNA that comprises a targeting sequence that binds a miRNA target site on the mRNA of human androgen receptor, operably linked to regulatory sequences which direct expression of the nucleic acid sequence in the subject, wherein the miRNA inhibits expression of human androgen receptor, and wherein the vector genome comprises
a) an AAV 5′ ITR;
b) a CMV enhancer;
c) a CBA promoter;
d) a chimeric intron;
e) SEQ ID NO: 4 or a sequence having up to 10 substitutions;
f) a woodchuck post-regulatory element (WPRE);
g) a poly A; and
h) an AAV 3′ ITR.

2. The AAV of claim 1, wherein the vector genome comprises one or more of
a) the AAV 5′ ITR of SEQ ID NO: 34;
b) the CMV enhancer of SEQ ID NO: 29;
c) the CBA promoter of SEQ ID NO: 30;
d) the chimeric intron of SEQ ID NO: 31;
e) SEQ ID NO: 4;
f) the woodchuck post-regulatory element (WPRE) of SEQ ID NO: 32;
g) the RBG poly A of SEQ ID NO: 33; or
h) the AAV 3′ ITR of SEQ ID NO: 35.

3. The AAV of claim 1, wherein the vector genome comprises the expression cassette of SEQ ID NO: 26, or a sequence sharing at least 80% identity therewith.

4. The AAV of claim 1, wherein the vector genome comprises SEQ ID NO: 28, or a sequence sharing at least 80% identity therewith.

5. A pharmaceutical composition comprising the AAV according to claim 1, and a pharmaceutically acceptable aqueous suspending liquid, excipient, and/or diluent.

6. A method for treating a subject having Spinal and Bulbar Muscular Atrophy (SBMA) comprising delivering an effective amount of the composition according to claim 5 to a subject in need thereof.

7. The method of claim 6, wherein the composition is formulated to be administered intrathecally at a dose of 1×10¹⁰ GC/g brain mass to 3.33×10¹¹ GC/g brain mass of the rAAV.

8. The method of claim 6, wherein the patient is a human adult and is administered a dose of 1.44×10¹³ to 4.33×10¹⁴ GC of the rAAV.

9. The method of claim 6, wherein the rAAV is delivered intrathecally, via intracerebroventricular delivery, or via intraparenchymal delivery.

10. The method of claim 6, wherein the composition is administered as a single dose via a computed tomography-(CT-) guided sub-occipital injection into the cisterna magna (intra-cisterna magna).

11. The method of claim 6, wherein the patient has SBMA.

12. The method of claim 6, wherein the patient is administered a dose of 1×10¹⁰ GC/g brain mass to 3.33×10¹¹ GC/g brain mass of the rAAV intrathecally.

13. The method of claim 6, wherein the patient is a human adult and is administered a dose of 1.44×10¹³ to 4.33×10¹⁴ GC of the rAAV.

14. An expression cassette comprising a nucleic acid sequence encoding at least one hairpin forming miRNA that comprises a targeting sequence that binds a miRNA target site on the mRNA of human androgen receptor, operably linked to regulatory sequences which direct expression of the nucleic acid sequence in the subject, wherein the miRNA inhibits expression of human androgen receptor, and wherein the expression cassette comprises
a) a CMV enhancer;
b) a CBA promoter;
c) a chimeric intron;
d) SEQ ID NO: 4 or a sequence having up to 10 substitutions;
e) a woodchuck post-regulatory element (WPRE); and
f) a poly A.

15. The expression cassette of claim 14, wherein the comprising one or more of
a) the CMV enhancer of SEQ ID NO: 29;
b) the CBA promoter of SEQ ID NO: 30;
c) the chimeric intron of SEQ ID NO: 31;
d) SEQ ID NO: 4;
e) the woodchuck post-regulatory element (WPRE) of SEQ ID NO: 32; or the RBG poly A of SEQ ID NO: 33.


SEQUENCE LISTING (35 sequences)

SEQ ID NO: 1 - DNA, 21 bp, Homo sapiens
  source 1..21: mol_type = other DNA
gaactacatcaaggaactcga

SEQ ID NO: 2 - DNA, 21 bp, synthetic construct
  misc_feature 1..21: note = constructed sequence
  source 1..21: mol_type = other DNA
tcgagttccttgatgtagttc

SEQ ID NO: 3 - DNA, 21 bp, synthetic construct
  misc_feature 1..21: note = constructed sequence
  source 1..21: mol_type = other DNA
cgatcgagttccttgatgtag

SEQ ID NO: 4 - DNA, 64 bp, synthetic construct
  misc_feature 1..64: note = constructed sequence
  source 1..64: mol_type = other DNA
tgctgtcgagttccttgatgtagttcgttttggccactgactgacgaactacaaaggaactcga

SEQ ID NO: 5 - DNA, 64 bp, synthetic construct
  misc_feature 1..64: note = constructed sequence
  source 1..64: mol_type = other DNA
tgctgcgatcgagttccttgatgtaggttttggccactgactgacctacatcagaactcgatcg

SEQ ID NO: 6 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 7 - DNA, 130 bp, synthetic construct
  misc_feature 1..130: note = constructed sequence
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 8 - DNA, 304 bp, synthetic construct
  misc_feature 1..304: note = constructed sequence
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 9 - DNA, 278 bp, synthetic construct
  misc_feature 1..278: note = constructed sequence
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 10 - DNA, 973 bp, synthetic construct
  misc_feature 1..973: note = constructed sequence
  source 1..973: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 11 - DNA, 131 bp, synthetic construct
  misc_feature 1..131: note = constructed sequence
  source 1..131: mol_type = other DNA
ctggaggcttgctgaaggctgtatgctgtcgagttccttgatgtagttcgttttggccactgactgacgaactacaaaggaactcgacaggacacaaggcctgttactagcactcacatggaacaaatggc

SEQ ID NO: 12 - DNA, 131 bp, synthetic construct
  misc_feature 1..131: note = constructed sequence
  source 1..131: mol_type = other DNA
ctggaggcttgctgaaggctgtatgctgcgatcgagttccttgatgtaggttttggccactgactgacctacatcagaactcgatcgcaggacacaaggcctgttactagcactcacatggaacaaatggc

SEQ ID NO: 13 - DNA, 589 bp, synthetic construct
  misc_feature 1..589: note = constructed sequence
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 14 - DNA, 130 bp, synthetic construct
  misc_feature 1..130: note = constructed sequence
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 460 bp, synthetic construct
  misc_feature 1..460: note = hSynapsin promoter
  source 1..460: mol_type = other DNA
tcgagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgt

SEQ ID NO: 16 - DNA, 8030 bp, synthetic construct
  misc_feature 1..8030: note = production plasmid pAAV2hu68n.KanR
  regulatory 1..36: note = promoter - Truncated P5; regulatory_class = promoter
  misc_feature 37..1899: note = AAV2 Rep
  regulatory 4220..4350: note = promoter - P5 promoter; regulatory_class = promoter
  regulatory 4398..4692: note = promoter - LacZ promoter; regulatory_class = promoter
  misc_feature 4875..5463: note = Col E1 ori
  misc_feature 6160..6975: note = KanR
  misc_feature 7195..7229: note = KanR
  misc_feature 7325..7780: note = f1 ori
  source 1..8030: mol_type = other DNA
  CDS 1919..4129: note = AAVhu68 Cap
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacactgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtct

SEQ ID NO: 17 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - DNA, 2211 bp, synthetic construct
  misc_feature 1..2211: note = hu68
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 19 - DNA, 2211 bp, synthetic construct
  misc_feature 1..2211: note = hu68 M191
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 20 - skipped

SEQ ID NO: 21 - protein, 743 aa, synthetic construct
  REGION 1..743: note = Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 2229 bp, synthetic construct
  misc_feature 1..2229: note = constructed sequence
  source 1..2229: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 23 - DNA, 841 bp, synthetic construct
  misc_feature 1..841: note = CB7 Promoter with CMV enhancer
  source 1..841: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctaga

SEQ ID NO: 24 - DNA, 1649 bp, synthetic construct
  misc_feature 1..1649: note = CAG promoter
  source 1..1649: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 25 - DNA, 127 bp, synthetic construct
  misc_feature 1..127: note = rabbit beta-globin
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 26 - DNA, 2701 bp, synthetic construct
  misc_feature 1..2701: note = Constructed sequence
  source 1..2701: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgctagctctggaggcttgctgaaggctgtatgctgtcgagttccttgatgtagttcgttttggccactgactgacgaactacaaaggaactcgacaggacacaaggcctgttactagcactcacatggaacaaatggcccagatctagggtggggccacctgccggtaggtgtgcggtaggcttttctccgtcgcaggacgcagggttcgggcctagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgaagctttctagagtcgacccgggcggcggcggccgcctaaggctcgaggatctttttccctctgccaaaaattatggggacatcatgaagccccttgagcatctgacttctggctaataaaggaaatttattttcattgcaatagtgtgttggaattttttgtgtctctcactcg

SEQ ID NO: 27 - DNA, 21 bp, Homo sapiens
  source 1..21: mol_type = other DNA
ctacatcaaggaactcgatcg

SEQ ID NO: 28 - DNA, 3154 bp, synthetic construct
  source 1..3154: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctgatatcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgctagctctggaggcttgctgaaggctgtatgctgtcgagttccttgatgtagttcgttttggccactgactgacgaactacaaaggaactcgacaggacacaaggcctgttactagcactcacatggaacaaatggcccagatctagggtggggccacctgccggtaggtgtgcggtaggcttttctccgtcgcaggacgcagggttcgggcctagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgaagctttctagagtcgacccgggcggcggcggccgcctaaggctcgaggatctttttccctctgccaaaaattatggggacatcatgaagccccttgagcatctgacttctggctaataaaggaaatttattttcattgcaatagtgtgttggaattttttgtgtctctcactcggataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - DNA, 304 bp, synthetic construct; identical to SEQ ID NO: 8
  source 1..304: mol_type = other DNA

SEQ ID NO: 30 - DNA, 278 bp, synthetic construct; identical to SEQ ID NO: 9
  source 1..278: mol_type = other DNA

SEQ ID NO: 31 - DNA, 973 bp, synthetic construct; identical to SEQ ID NO: 10
  source 1..973: mol_type = other DNA

SEQ ID NO: 32 - DNA, 589 bp, synthetic construct; identical to SEQ ID NO: 13
  source 1..589: mol_type = other DNA

SEQ ID NO: 33 - DNA, 127 bp, synthetic construct; identical to SEQ ID NO: 25
  source 1..127: mol_type = other DNA

SEQ ID NO: 34 - DNA, 130 bp, synthetic construct; identical to SEQ ID NO: 7
  source 1..130: mol_type = other DNA

SEQ ID NO: 35 - DNA, 130 bp, synthetic construct; identical to SEQ ID NO: 14
  source 1..130: mol_type = other DNA